(19) United States
(12) Patent Application Publication
(10) Pub. No.: US 2021/0072213 A1
(43) Pub. Date: Mar. 11, 2021

(54) POLY(DIACETYLENE) SENSOR ARRAYS FOR CHARACTERIZING AQUEOUS SOLUTIONS

(71) Applicant: AARHUS UNIVERSITET, Aarhus C (DK)

(72) Inventors: Brigitte Maria STÄDLER, Aarhus N (DK); Järvi SPANJERS, Wijchen (NL); Fabian Robert ITEL, Teufen (CH)

(21) Appl. No.: 16/959,461

(22) PCT Filed: Jan. 3, 2019
(86) PCT No.: PCT/DK2019/050001

§ 371 Date: Jul. 1, 2020

(30) Foreign Application Priority Data
Jan. 3, 2018 (DK) PA 2018 70003

(51) Int. Cl. G01N 33/14; C08F 38/00; G01N 31/22

(52) U.S. Cl. CPC G01N 33/146; G01N 31/22; C08F 38/00

(57) ABSTRACT

The present invention relates to colorimetric polydiacetylene (PDA) sensor arrays for detection of analytes and levels thereof in aqueous solutions. In particular the present invention relates to methods of characterizing an aqueous solution for at least one analyte, comprising the steps of a) providing a sensor array comprising at least two different poly-diacetylenes, wherein said poly-diacetylenes are spatially separated and individually addressable, b) contacting said sensor array with a sample of said aqueous solution, c) measuring the colorimetric response of said poly-diacetylenes to the aqueous solution, wherein said poly-diacetylenes are polymerized from a composition comprising one or more diacetylene monomer(s) said poly-diacetylenes are capable of a colorimetric response upon contact with said analyte, and wherein the at least one analyte is selected from the group consisting of an organic molecule with a molecular weight below 2000 g/mol, salts thereof and an inorganic salt.

### TECHNICAL FIELD OF THE INVENTION

The present invention relates to colorimetric polydiacetylene (PDA) sensor arrays for detection of analytes an levels thereof in aqueous solutions. In particular the present invention relates to the detection of analytes and levels thereof present in beverages such as beer and beer precursors using said sensors.

### BACKGROUND OF THE INVENTION

Methods for fast and reliable flavour detection from complex mixtures such as dairy products or alcoholic and non-alcoholic beverages are of interest for product development, quality and safety.

Today's dominant approaches remain rather complex and labour intense focusing on gas chromatography and/or sensory panels. Electronic tongue sensors employing artificial membranes and electrochemical techniques, are an emerging concept but many technical, material and computational challenges need to be tackled before they can become widely applicable. Alternative approaches which would allow for fast on-site screening are therefore in high demand. Especially interesting in this context are colorimetric sensors for instance based on poly(diacetylenes) (PDA). Diacetylene (DA) monomers can be polymerized into PDA, typically a blue coloured polymer, within a few minutes without the need of a catalyst or an initiator. In response to various external stimuli including temperature, solvents exposure, or ligand-receptor interactions PDA undergoes a blue-to-red (and non-fluorescent-to-fluorescent) configuration shift which is easy detectable. PDA sensors in the form of vesicles, embedded into electrospun fibres, connected to carbon nanotubes, inorganic porous materials, or paper among others were reported.

EP 2947455 A1 discloses a hydrochromic polydiacetylene (PDA)-cation composite composition and a hydrochromic thin film of the PDA composite composition reacting sensitively to moisture. The use of PCDA (10,12-pentacosadiynoic acid), TCDA (10,12-tricosadiynoic acid), HCDA (8,10-heneicosadiynoic acid) in the preparation of PDA composites is disclosed. The, PDA composite is thus polymers of the above acids with an alkali counter ion such as Li+, Na+, K+, Rb+, Cs+[0014]-[0016]. An array of spatially separated PDA's for characterising aqueous compositions comprising analytes via colorimetric measurement is not disclosed.

US 2016/0061741 A1 discloses PDA and PDA/ZnO nanocomposites based on the monomers PCDA, TCDA and DCDA [0008], and their application as chemical sensing agents for selected organic liquids e.g. methanol, ethanol, benzyl alcohol, octanol, diethyl ether, DMF, DCM THF and acetone [0073]. An array of spatially separated PDA's which allows for characterisation of aqueous solutions comprising analytes is not disclosed.

EP 1161688 B1 discloses an aggregate particle comprising lipids and a polymer [0005]. The polymers may be diacetylene acids and diacetylene derivatives such as tricosadiynoic acid (TCDA), tricosadiynoic methyl esters, pentacosadiynoic acid (PCDA) and pentacosadiynoic methyl esters. The lipids disclosed are preferably phospholipids [0011]. The aggregate particle may be used for detection of peptides (native peptides) or analogues thereof by providing a colour shift in the presence of the peptide [0013]. An array of spatially separated PDA's which allows for characterisation of aqueous solutions comprising analytes is not disclosed.

Eaidkong T. et al., J. Mater. Chem., 2012, 22, 5970 disclose the use of paper-based PDAs as colorimetric sensors prepared from eight diacetylene monomers including PCDA and TCDA (abstract and FIG. 1). The array is used for vapour phase detection of volatile organics from automotive fuels. The characterisation of aqueous solutions, such as beverages, by measurement in the aqueous phase, is not disclosed.

### SUMMARY OF THE INVENTION

Although various application for PDA sensors were considered, their use in the context of food and beverage safety, development and process monitoring remains largely unexplored. Hence, a PDA sensor for fast, cheap and reliable on-site characterization and/or detection of analytes in aqueous solutions would be advantageous. Particularly, a PDA sensor array capable of providing a fingerprint type identification of a beverage or beverage precursor and capable of rapidly distinguishing between e.g. two distinctive beverage batches or brands would be advantageous. It would be particularly advantageous to compare the colorimetric response of identical arrays for a test batch with the response of a reference batch to e.g. determine that the test batch is similar to the reference batch.

Thus, an object of the present invention relates to providing a PDA sensors array for fast and reliable characterization and/or detection of analytes or levels thereof in aqueous solutions, in particular in complex aqueous solutions, such as beverages, for example dairy or beer. In particular, it is an object of the present invention to provide a PDA sensor array that solves the above mentioned problems of the prior art of providing a reliable and fast method of characterising and/or distinguishing between aqueous solutions comprising analytes of interest, such as for example flavour constituents in beer.

Thus, one aspect of the invention relates to a method for characterizing an aqueous solution for at least one analyte, comprising the steps of
a) providing a sensor array comprising at least two different poly-diacetylenes, wherein said poly-diacetylenes are spatially separated and individually addressable,
b) contacting said sensor array with a sample of said aqueous solution,
c) measuring the colorimetric response of said poly-diacetylenes to the aqueous solution,
   wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkylene, an optionally substituted C₂-C₃₀ alkenylene, and an optionally substituted C₂-C₃₀ alkynylene, wherein
   said poly-diacetylenes are capable of a colorimetric response upon contact with said analyte, and wherein
   the at least one analyte is selected from the group consisting of an organic molecule with a molecular weight below 2000 g/mol, salts thereof and an inorganic salt.

Another aspect of the present invention relates to a method for characterizing a beer or a beer precursor for multiple analytes, comprising the steps of
a) providing a sensor array comprising at least two different poly-diacetylenes, wherein said poly-diacetylenes are spatially separated and individually addressable,
b) contacting said sensor array with a sample of a beer or a beer precursor,
c) measuring the colorimetric response of said poly-diacetylenes to the beer or beer precursor, and
   wherein said poly-diacetylenes are polymers polymerised from a composition comprising a diacetylene monomer or mixtures thereof, and
   wherein said sensor array for each analyte comprises at least one poly-diacetylene capable of a colorimetric response to contact to said analyte, and
   wherein the analytes are flavour constituents of beer.

Another aspect of the invention is a method for comparing a test aqueous solution with a reference aqueous solution comprising at least one analyte, comprising the steps of
a) providing at least two identical sensor arrays comprising at least two different poly-diacetylenes, wherein said poly-diacetylenes are spatially separated and individually addressable,
b) contacting a first sensor array with a sample of the test aqueous solution and a second sensor array with a reference aqueous solution,
c) comparing the colorimetric response of said poly-diacetylenes of the first sensor array to the colorimetric response of said poly-diacetylenes of the second sensor array,
   wherein a similar colorimetric response of the first sensor array and the second sensor array indicates that the test aqueous solution is similar to the reference aqueous solution; and
   wherein said poly-diacetylenes are polymers polymerised from a composition comprising a diacetylene monomer or mixtures thereof.

Yet another aspect of the present invention relates to a sensor array comprising at least two different poly-diacetylenes,

wherein said poly-diacetylenes are spatially separated and individually addressable, and
wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, and
wherein said poly-diacetylenes are capable of a colorimetric response upon contact with an analyte.

The present inventors have surprisingly found that using the above methods they are able to characterise and even distinguish between aqueous solutions that are very closely related, such as for example closely related beverages. A method capable of distinguishing for example four different commercial beers was thus demonstrated with the use of just a few different diacetylene monomers and by measuring on just a few analytes in these beers.

### BRIEF DESCRIPTION OF THE FIGURES

FIG. 1a-f shows time dependent change of the RGB intensity for sensors fabricated from H/T (1:1 mol-ratio) (a), H (b), P/T (1:1 mol-ratio) (c), P (d), H/P (1:1 mol-ratio) (e), and T (f) exposed to the lab environment. [T=10,12-tricosadiynoic acid (98%), P=10,12-pentacosadiynoic acid (97%), H=5,7-hexadecadiynoic acid (97%)].

FIG. 2a-c shows RGB colour change profile of paper-based PDA sensor arrays consisting of T, P and H as well as their 3/1, 1/1 and 1/3 vol-ratio mixtures after exposure to 100% EtOH (a), 10% EtOH (b) and 100 H₂O (c); FIG. 2d shows corresponding PC score plots of the RGB colour changes obtained from the same sensor arrays.

FIG. 3a-c shows: RGB colour change profile of paper-based PDA sensor arrays consisting of T and P as well as their 1/1 volume-ratio mixtures fabricated from different DA monomer concentrations, after exposure to 100% EtOH (a), 10% EtOH (b) and 100 H₂O (c); FIG. 3d shows corresponding PC score plots of the RGB colour changes obtained from the sensor arrays (n=3).

FIG. 4a-d shows RGB colour change profile of paper-based PDA sensor arrays consisting of T and P as well as their 1/1 volume-ratio mixtures fabricated from different monomer concentrations, after exposure to a 2.5% EtOH (a), 5% EtOH (b), 10% EtOH (c) and 15% EtOH (d) solution; FIG. 4e shows corresponding PC score plots of the RGB colour changes obtained from the sensor arrays (n=3).

FIG. 5a-d shows RGB colour change profile of paper-based PDA sensor arrays consisting of T and P as well as their 1/1 volume-ratio mixtures fabricated from different monomer concentrations, after exposure to a 5% EtOH (a), or a 5% EtOH solution supplemented with 2 ppm (b), 19 ppm (c) or 155 ppm ethyl acetate; FIG. 5e shows corresponding PC score plots of the RGB colour changes obtained from the sensor arrays.

FIG. 6a-d shows RGB colour change profile of paper-based PDA sensor arrays consisting of T and P as well as their 1/1 volume-ratio mixtures fabricated from different monomer concentrations, after exposure to a 5% EtOH (a), or a 5% EtOH solution supplemented with 2 ppm (b), 16 ppm (c) or 155 ppm diacetyl; FIG. 6e shows corresponding PC score plots of the RGB colour changes obtained from the sensor arrays.

FIG. 7a-d shows RGB colour change profile of paper-based PDA sensor arrays consisting of T and P as well as their 1/1 volume-ratio mixtures fabricated from different monomer concentrations, after exposure to a 5% EtOH (a), or a 5% EtOH solution supplemented with 2 ppm (b), 19 ppm (c) or 186 ppm (d) acetylpropionyl.; FIG. 7e shows corresponding PC score plots of the RGB colour changes obtained from the sensor arrays.

FIG. 8a-d shows RGB colour change profile of paper-based PDA sensor arrays consisting of T and P as well as their 1/1 volume-ratio mixtures fabricated from different monomer concentrations, after exposure to 4 different commercial beers: Carlsberg Nordic (CB N), Tuborg Classic (TB C), Carlsberg Classic (CB C) and WIIBROE (WB);

FIG. 8e shows corresponding PC score plots of the RGB colour changes obtained from the sensor arrays for Carlsberg Nordic (CB N), Tuborg Classic (TB C), Carlsberg Classic (CB C) and WIIBROE (WB).

FIG. 8f-g shows sensor arrays colours for 4 different commercial beers: Carlsberg Nordic (CB N), Tuborg Classic (TB C), Carlsberg Classic (CB C) and WIIBROE (WB). FIG. 9a shows colour differences for the 4 beers for different PDA sensor types or combinations thereof (T, T/P, P, P/H, H, H/T), while FIG. 9b shows colour differences between beers at different polymer concentrations for T, T/P and P.

FIG. 9 shows a schematic overview of an example of vesicle and nanoparticle formation from DA monomers as used in solution to provide a solutions based assay.

FIGS. 10a-b shows results in the form of the colorimetric response of solution based detection of alcohols, esters and 4-VG (10a), and particularly 4-VG (10b) with sensors made in accordance with example 9. The tested sensors (mixtures of sensor 3, 14 and 18 of Table 2) show sensitivity to analytes (10a) and the ability to measure the presence of 4-VG in the presence of other analytes (10b) mimicking the beer environment.

FIG. 11a-b shows how pie charts showing differences in red chromaticity shift (RCS, 11a)) or hue (11b) values may identify, characterize and allow comparison of reference arrays with test arrays to determine whether one beer is similar/the same or different from another beer. 4 different commercial beers are shown to differ in this RCS assay with 10 sensors per array. Numbers on pie charts refer to different paper sensors in accordance with Tables 2 and 3 of the examples. A darker colour indicates a higher RCS or hue value (from 0-100) as calculated in accordance with the method indicated in the examples.

FIG. 12a-d shows the synthetic procedures of DA monomers 4, 14, 18 and 8 (Table 2) as representative examples of the set of monomers shown in that table.

The present invention will now be described in more detail in the following.

### DETAILED DESCRIPTION OF THE INVENTION

### Definitions

Prior to discussing the present invention in further details, the following terms and conventions will first be defined:

Aqueous solution

In the present context an aqueous solution in the broadest sense is any liquid comprising water in any amount. It includes homogenous solutions or mixtures and inhomogeneous mixtures such as dispersions or emulsions of e.g. fats in water (for example dairy milk). Particularly, the aqueous solutions of the present invention may comprise complex mixtures of many analytes and additional components in water. Aqueous solution may be used interchangeably with aqueous compositions.

### Analyte

In the present context an analyte in the broadest sense is any compound or entity capable of interacting with the sensor array of the invention. An analyte may or may not be present in the sample of the aqueous solution of the present invention. Analytes may be dissolved, dispersed, or part of an emulsion.

Sensor array

In the present context a sensor array is a solid support comprising a plurality (two or more) of spatially separated sensors capable of interacting with analytes of interest. Particularly, in the sensor arrays of the present invention the sensors comprise the polydiacetylenes of the invention in amounts sufficient to produce a measurable colorimetric response.

Diacetylene monomer

In the present context a diacetylene monomer is the monomer (or monomers) used in a polymerisation process to produce polydiacetylenes. A diacetylene group consisting of two acetylene groups separated by a single bond (R′—≡—≡—R″) is comprised in such monomers. The monomers may include several diacetylene groups, which facilities cross coupling and thus non-linear polydiacetylenes.

### Poly(diacetylene)

In the present context a polydiacetylenes is a polymer obtained from polymerisation of diacetylene monomers. They may be represented by the general formula (A) below, when a single diacetylene monomer with only one diacetylene moiety (R′—≡—≡—R″) is used during polymerisation.

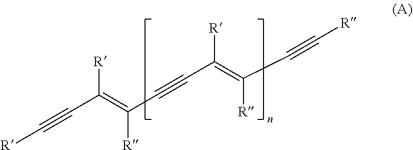

Such polymerisation results in a linear polymer with R′ and R″ groups distributed evenly along the polymer chain. When a mixture of two or more different monomers are used, the R groups may vary along the polymer chain randomly. Also, if the monomer comprises more than one diacetylene group (e.g. if R′ and/or R″ comprises a further diacetylene), cross coupling will occur and non-linear polymers or polymer matrices may be obtained.

Organic molecule

In the present context organic molecule has its usual meaning. It does not include large macromolecules or polymers, but may include salts or free bases or acids of organic molecules as well as molecules binding metal ions (chelates). Relevant sub-groups include small molecules below a certain molecular weight threshold and, volatile organic compounds (VOCs), and flavour molecules, particularly beer flavour constituents.

Inorganic salt

In the present context inorganic salt has its usual meaning and is the combination of a cationic species and an anionic species. It may further include free ions, as these may in some cases bind to the polydiacetylenes sensors without a counter ion present.

### Characterizing

In the present context characterizing has its usual meaning and involves obtaining a set of data that enables the characterisation of an aqueous composition comprising one or more analytes. Characterising may be used interchangeably with identifying. Preferably the characterisation is able to provide a data set which is unique for the specific aqueous composition of analytes in the sense, that any changes to analyte amounts or presence of further measurable analytes will provide a measurably different results. In other words the characterisation is ideally able to distinguish between aqueous solution having different analyte content and/or different levels of analyte comprised.

Optionally substituted

In the present context “optionally substituted” means that a chemical moiety or group may or may not be substituted with one or a plurality of compatible substituents known in the field of organic synthesis. In the present context “substituted” means that a chemical moiety or group has one or more substituents (further chemical moiety or group) attached in addition to those implied by the name of the moiety or group.

Alkylene, alkenylene, alkynylene

In the present context alkylene, alkenylene, alkynylene have their usual meaning, i.e. they represent hydrocarbon chains, where alkylenes comprise single bonds only, where alkenylene chains comprise at least one carbon-carbon double bond, and alkynylene comprises at least one carbon-carbon triple bond. The hydrocarbon chains may be straight or branched. The chains are open-ended, i.e. as represented by for example —(CH₂)ₙ—, n being an integer.

Flavour constituent

In the present context a flavour constituent is any molecule or salt capable of contributing to the flavour of e.g. a beverage, i.e. capable of interacting with the human or animal flavour detection system. Particular beverages such as beer, ciders and wine have particular flavour constituents known to the skilled individual. Flavour constituents may particularly comprise organic compounds, salts thereof and inorganic salts.

Beverage and precursors thereof

In the present context a beverage is an aqueous composition for human consumption comprising analytes, which are typically flavour constituents of the beverage. Precursors of beverages are aqueous intermediate products at any stage in the production line, prior to arriving at the final product (the beverage).

Amino acid

In the present context amino acids in the broadest sense are any natural or synthetic amino acid that may be present in the analysed solutions. This includes proteinogenic amino acids but also natural and synthetic derivatives thereof.

Colorimetric response

In the present context a colorimetric response is a measurable colour change in one or more of the poly-diacetylenes present on the sensor array induced by one or more analytes in the analysed aqueous solutions. The colour change may be compared to a reference array (optionally subjected to a reference solution), or compared to the same array prior to subjection to a sample solution. The colour change may be in the visible spectrum, but may also extend into the infrared and ultraviolet spectra. A colorimetric response may also be a colour difference between two or more corresponding poly-diacetylenes on each their array, which have been subjected to different sample solutions. There are several sub-types of colorimetric responses as described below and in the examples.

In one embodiment, the colorimetric response may be determined by determining the RBG value or the absorbance of each sensor before and after contact with the aqueous solution. In embodiments where the sensor is positioned on a solid support, e.g. on paper, the colour may e.g. be determined with the aid of a scanner, whereas a spectrophotometer may be used when the sensor is in solution. The colorimetric response may then be determined as a change in RGB value (ARGB) or a change in absorbance.

In one embodiment the colorimetric response is determined by determining the RGB or several sensors before and after contact with the aqueous solution, and analysing the RGB values by standard statistical methods, for example by a principal component analysis. The PCA may e.g. be used to determine a cluster mean, which can be used as an indication of the colorimetric response. This may for example be done as described in the Examples below in the section “Detection”. Closeness in space of the cluster mean indicates that two aqueous solutions are similar.

In one embodiment, the colorimetric response may be determined by calculating the percent change in the percentage of a particular colour (e.g. percentage of red, green or blue) based on the RGB value. The colorimetric response for percentage blue (CR_{blue}) may for example be determined by determining light absorbance at two specific wavelengths (e.g. at 640 nm and 548 nm) of each sensor before and after contact with the aqueous solution and then calculating the percent change in the percentage of a particular absorbance.

In one embodiment the colorimetric response is determined by determining the red chromaticity shift (RCS) as defined in the examples in the section “Detection”. In one embodiment the colorimetric response is determined by determining a change in the hue value as defined in the examples in the section “Detection”.

### METHODS OF THE INVENTION

The present inventors have developed a method involving a polydiacetylenes based sensor array which is capable of characterising complex aqueous solutions by colorimetric measurements.

Thus, a first aspect of the present invention is a method for characterizing an aqueous solution for at least one analyte, comprising the steps of
a) providing a sensor array comprising at least two different poly-diacetylenes, wherein said poly-diacetylenes are spatially separated and individually addressable,
b) contacting said sensor array with a sample of said aqueous solution,
c) measuring the colorimetric response of said poly-diacetylenes to the aqueous solution,
   wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, wherein
   said poly-diacetylenes are capable of a colorimetric response upon contact with said analyte, and wherein
   the at least one analyte is selected from the group consisting of an organic molecule with a molecular weight below 2000 g/mol, salts thereof and an inorganic salt.

Another aspect of the invention is a method for comparing a test aqueous solution with a reference aqueous solution comprising at least one analyte, comprising the steps of
a) providing at least two identical sensor arrays comprising at least two different poly-diacetylenes, wherein said poly-diacetylenes are spatially separated and individually addressable,
b) contacting a first sensor array with a sample of the test aqueous solution and a second sensor array with a reference aqueous solution,
c) comparing the colorimetric response of said poly-diacetylenes of the first sensor array to the colorimetric response of said poly-diacetylenes of the second sensor array,
   wherein a similar colorimetric response of the first sensor array and the second sensor array indicates that the test aqueous solution is similar to the reference aqueous solution; and
   wherein said poly-diacetylenes are polymers polymerised from a composition comprising a diacetylene monomer or mixtures thereof.

In the present context ‘identical sensor arrays’ are defined as essentially identical in the sense that they are produced by analogous methods and with the same polydiacetylenes monomers and ratios thereof for each sensor in the array.

In the present context the term “similar solutions” is defined as solutions that after e.g. colorimetric analysis of the first and second sensor array the results are comparable within a given threshold as readily defined by the skilled person. For example, whether two sensors have been subjected to near identical or similar solutions may be determined via closeness in space in a principal component (PC) analysis plot. Thus, the closer in space the cluster mean obtained by a PCA of the RGB values determined before and after contact with two different aqueous solutions, the more similar these two aqueous solutions are considered to be. Alternatively, the arrays may be compared in terms of one or more of the following parameters: Percentage change of a particular colour (e.g. CR_{blue}), ARGB, red chromaticity shift (RCS), and/or Hue values for each PDA sensor. Two aqueous solutions are considered similar if the difference in one or more of these parameters are lower than a predetermined threshold. Similarly, two aqueous solutions are considered different if the difference in one or more of these parameters are higher than a predetermined threshold. For example a similar colorimetric response may be a colorimetric response value within 10% for each sensor, such as within 8%, such as 6%, 4%, 3%, 2%_{, 1}%, 0.5%, such as preferably 0.1% for each sensor.

The diacetylene monomers of the present invention are polymerised in solution by activation e.g. by subjecting them to radiation, such as UV-radiation. The polydiacetylenes formed may be formed from a single monomer or a mixture or two or more different monomers. Monomers or mixtures thereof comprising a single diacetylene moiety will form a linear polymer, whereas if further diacetylene monomers are comprised, such as in a C₂-C₃₀ alkynyl group, cross-linked polymers, such as polymer matrices may be formed. Thus, in one embodiment of the invention the optionally substituted C₂-C₃₀ alkynyl comprises further diacetylene groups, such as one further diacetylene group or a plurality of further diacetylene groups.

The diacetylene monomers may in a preferred embodiment be substituted with a polyethylene glycol alkyl ether. Alternatively, the diacetylene monomers may in a preferred embodiment be substituted with a. optionally substituted imidazolium. Such groups may e.g. improve solubility of the monomers.

The diacetylene monomers of the invention may comprise the optionally substituted C₁-C₃₀ alkyl, optionally substituted C₂-C₃₀ alkenyl, and/or an optionally substituted C₂-C₃₀ alkynyl groups in the form of alkylene, alkenylene, or alkynylene groups attached to the diacetylene moiety and an end-group respectively. Thus in another embodiment of the invention said one or more diacetylene monomer(s) is selected from the group of diacetylenes according to formula (I) or (II)

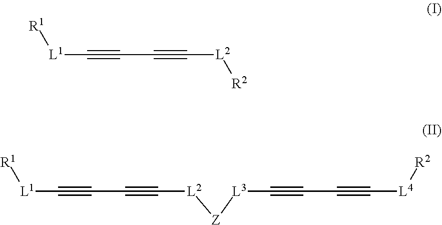

or mixtures thereof, wherein
L¹, L², L³ and L⁴ are the same or different and individually selected from the group consisting of an optionally substituted C₁-C₃₀ alkylene, an optionally substituted C₂-C₃₀ alkenylene, and an optionally substituted C₂-C₃₀ alkynylene,
R¹ and R² are the same or different and individually selected from the group consisting of —CH₃, OR³, SR³, —COOR³, —CONR⁴R⁵, wherein R³, R⁴, and R⁵ are individually selected from the group consisting of hydrogen, C₁-C₈ alkyl optionally substituted with a thiol, vinyl, an amino acid, or optionally substituted imidazolium, and a polyethylene glycol alkyl ether optionally substituted with a thiol, vinyl, an amino acid, or optionally substituted imidazolium, or are selected so that NR⁴R⁵ constitutes an amino acid,
Z is selected from the group consisting of optionally substituted alkylene, aryl, —CONH—(CH₂)x-HNCO— where X is an integer between 1 and 20, and heteroaryl.

The length of the L group may vary both on the individual monomers of formula (I) and (II), and also in the different monomers used, and thus in one embodiment of the invention L¹, L², L³ and L⁴ are the same or different and individually selected from the group consisting of C₁-C₂₀, such as C₁-C₁₈, such as C₁-C₁₅, such as C₂-C₁₂ optionally substituted alkylene, alkenylene and alkynylene. L¹, L², L³ and L⁴ may also be the same or different and individually selected from a —(CH₂)ₙ— group wherein n is 1-30, such as 1-20, 1-18, 1-15, such as preferably 1-12.

The present inventors have found that variation of the chain length in the individual monomers (e.g. between the chain length of L¹ and one or more of either L², L³ and/or L⁴) provides for good characterisation of analytes due to variations in the resulting colorimetric responses to the individual analytes.

Various combinations of chain lengths in the monomers may be utilised to provide optimum characterisation towards particular compositions or analytes.

Advantageous combinations of chain lengths for L¹ versus L² or L³ versus L⁴ may preferably be C₁-C₂₀ versus C₁₀-C₃₀, such as C₁-C₁₀ versus C₁₀-C₂₀, C₂-C₈ versus C₅-C₁₅, C₂-C₈ versus C₈-C₁₅, C₂-C₆ versus C₄-C₁₂, such as C₂-C₆ versus C₆-C₁₂.

Thus, in a particular embodiment at least one of L1 or L³ in the diacetylene according to formula (I) or (II) is different from at least one of L² and L⁴, and at least one of L¹ or L³ is a C₁-C₁₅ optionally substituted alkylene, alkenylene or alkynylene, and at least one of L² and L⁴ is a C₁₆-C₃₀ optionally substituted alkylene, alkenylene or alkynylene. Alternatively at least one of L¹ or L³ in the diacetylene according to formula (I) or (II) is different from at least one of L² and L⁴, and at least one of L¹ or L³ is a C₁-C₁₀ optionally substituted alkylene, alkenylene or alkynylene, and at least one of L² and L⁴ is a C₁₁-C₂₀ optionally substituted alkylene, alkenylene or alkynylene. Alternatively at least one of L1 or L³ in the diacetylene according to formula (I) or (II) is different from at least one of L² and L⁴, and at least one of L¹ or L³ is a C₁-C₈ optionally substituted alkylene, alkenylene or alkynylene, and at least one of L² and L⁴ is a C₉-C₁₅ optionally substituted alkylene, alkenylene or alkynylene. Alternatively at least one of L1 or L³ in the diacetylene according to formula (I) or (II) is different from at least one of L² and L⁴, and at least one of L¹ or L³ is a C₅-C₈ optionally substituted alkylene, alkenylene or alkynylene, and at least one of L² and L⁴ is a C₉-C₁₂ optionally substituted alkylene, alkenylene or alkynylene.

The monomer end-groups R¹ and R² may be selected to be simply a methyl group, or they may be selected from functional groups capable of interaction with specific analytes or groups thereof. For example if an analyte of interest comprises a vinyl group, an end group reactive to a vinyl group may be used in one or more diacetylene monomers

More specifically, as described above R¹ and R² may be the same or different and may be individually selected from the group consisting of —CH₃, —OR³, —SR³, —COOR³, —CONR⁴R⁵, wherein

R³, R⁴, and R⁵ are individually selected from the group consisting of hydrogen, C₁-C₈ alkyl optionally substituted with a thiol, vinyl, an amino acid, optionally substituted imidazolium, and a polyethylene glycol alkyl ether optionally substituted with a thiol, vinyl, an amino acid, or optionally substituted imidazolium, or are selected so that NR⁴R⁵ constitutes an amino acid.

The polyethylene glycol alkyl ether may preferably be polyethylene glycol methyl, ethyl or propyl ether, particularly methyl ether.

The amino acid may particularly be selected from the group consisting of Histidine, Isoleucine, Leucine, Lysine, Methionine, Phenylalanine, Threonine, Tryptophan, Valine, Arginine, Cysteine, Glutamine, Glycine, Proline, Serine, Tyrosine, Alanine, Asparagine, Aspartic acid, Glutamic acid. The amino acid may preferably be arginine. The polyethylene glycol (PEG) alkyl ether may comprise 1-PEG units.

In one preferred embodiment R¹ and R² are the same or different and individually selected from the group consisting of —CH₃, and —COOR³. In another preferred embodiment R³, R⁴, and R⁵ are individually selected from the group consisting of hydrogen, and C₁-C₃ alkyl.

The Z group of formula (II) may be any group capable of forming a link between the two diacetylene moieties. Such groups may include optionally substituted alkylene, aryl, —CONH—(CH₂)x-HNCO— where X is an integer between 1 and 20, and heteroaryl groups. One such group may include ortho-dihydroxy terephthalic acid where L²/L³ are attached via an ether linkage at the two hydroxy groups.

In a particularly preferred embodiment of the present invention the substituents as provided in Formulas (I) and (II) are selected as follows:

L¹, L², L³ and L⁴ are the same or different and individually selected from a —(CH₂)ₙ— group wherein n is an integer in the range of 1-20,
R¹ and R² are the same or different and individually selected from the group consisting of —CH₃, —COOR³, —CONR⁴R⁵, wherein R³, R⁴, and R⁵ are individually selected from the group consisting of hydrogen, and C₁-C₈alkyl optionally substituted with a thiol, vinyl, an amino acid, or optionally substituted imidazolium, and a polyethylene glycol alkyl ether optionally substituted with a thiol, vinyl, an amino acid, or optionally substituted imidazolium, or are selected so that NR⁴R⁵ constitutes an amino acid, and
Z is selected from the group consisting of optionally substituted alkylene, aryl, —CONH—(CH₂)x-HNCO— where X is an integer between 1 and 20, and heteroaryl.

In one embodiment of the present invention one or more diacetylene monomer(s) is selected from the group of diacetylenes according to formula (I), wherein

L¹ is a —(CH₂)ₙ— group wherein n is an integer in the range of 1 to 20, for example in the range of 1 to 10, such as in the range of 1 to 8, for example in the range of 1 to 6; and
L² is a —(CH₂)ₙ— group wherein n is an integer in the range of 1 to 20, for example in the range of 10 to 20, such as in the range of 5 to 15, for example in the range of 8 to 15, such as in the rage of 4 to 12, for example in the range of 6 to 12; and
R¹ and R² are the same or different and individually selected from the group consisting of —CH₃, —COOR³, —CONR⁴R⁵, wherein R³, R⁴, and R⁵ are individually selected from the group consisting of hydrogen, and C₁-C₈ alkyl optionally substituted with a thiol, vinyl, an amino acid, or optionally substituted imidazolium, and a polyethylene glycol alkyl ether optionally substituted with a thiol, vinyl, an amino acid, or optionally substituted imidazolium, or are selected so that NR⁴R⁵ constitutes an amino acid.

Particularly preferred diacetylene monomers are listed in the table 1 below.

TABLE 1
preferred diacetylene monomers:

| Formula | R¹ | L¹ | L² | R² |
|---|---|---|---|---|
| (I) | —CH₃ | C₁-C₃₀ | C₁-C₃₀ | —COOH |
| | | alkylene | alkylene | |
| | —CH₃ | C₁-C₃₀ | C₁-C₃₀ | —COOMe |
| | | alkylene | alkylene | |
| | —CH₃ | C₁-C₃₀ | C₁-C₃₀ | —COOEt |
| | | alkylene | alkylene | |
| | —CH₃ | C₁-C₃₀ | C₁-C₃₀ | —OH |
| | | alkylene | alkylene | |
| | —CH₃ | C₁-C₃₀ | C₁-C₃₀ | —COO(EtO)_{y}—R³ |
| | | alkylene | alkylene | Y is 1-20, R³ is as defined above |
| | —CH₃ | C₁-C₃₀ alkylene | C₁-C₃₀ alkylene | 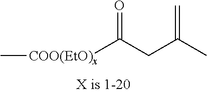 |
| | —CH₃ | C₁-C₃₀ | C₁-C₃₀ | —CONH(CH2)ₓ—SH |
| | | alkylene | alkylene | X is 1-10 |
| | —CH₃ | C₁-C₃₀ | C₁-C₃₀ | —CO—(N-amino acid) |
| | | alkylene | alkylene | |
| | —CH₃ | C₁-C₃₀ alkylene | C₁-C₃₀ alkylene | 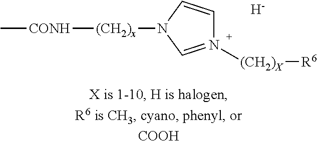 |
| | —CH₃ | C₁-C₃₀ alkylene | C₁-C₃₀ alkylene | 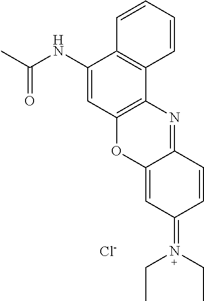 |
| | R¹/R² | L¹/L⁴ | L²/L³ | Z |
| (II) | —CH₃ | C₁-C₃₀ | C₁-C₃₀ | —CONH—(CH₂)ₓ—HNCO— |
| | | alkylene | alkylene | X is 1-10 |

In a particularly preferred embodiment the diacetylene monomers are selected from the group consisting of 5,7-hexadecadiynoic acid, 10,12-tricosadiynoic acid, and 10,12-pentacosadiynoic acid or mixtures thereof.

The PDA's of the array of the invention are defined by the monomers used to form them and the conditions at which they are polymerised. The present inventors have found that mixed PDA polymers, which are polymerized from two or more different diacetylene monomers are particularly advantageous in the characterisation of aqueous compositions. Hence, in one embodiment at least one of the poly-diacetylenes is a polymer polymerized from a mixture comprising at least two different diacetylene monomers.

The conditions used during polymerisation may also influence the performance of the array, and may be varied depending on analytes and monomers used. Particularly the concentration of monomer used during polymerisation on e.g. a solid support may influence the colorimetric response. Thus, particularly for solid supports such as paper, the concentration of diacetylene monomer or mixture thereof during polymerisation may be in the range of 1-1000 mM, such as 2-500 mM, 5-200 mM, 8-150 mM, 10-100, such as preferably 20-75 mM.

For any given aqueous composition comprising a number of analytes, some of which may be known in advance, it may be particularly advantageous to provide polydiacetylenes on the array, which are capable of a colorimetric response in the presence of particular analytes. Therefore, in a preferred embodiment the method is a method for characterizing an aqueous solutions for at least a first analyte and a second analyte, and wherein at least one poly-diacetylene is capable of a colorimetric response upon contact with said first analyte and at least one poly-diacetylene is capable of a colorimetric response upon contact with said second analyte. Similarly, the method may be a method for characterizing an aqueous solutions for multiple analytes, and wherein said sensor array for each analyte comprises at least one poly-diacetylene capable of a colorimetric response to contact to said analyte. Thus, the method may be a method for characterising at least 3 analytes, for example at least 5 analytes, such as in the range of 2 to 20 analytes.

Preferably, the PDA's of the present invention should not only respond to the presence of a given analyte but should also respond differently depending on what level (concentration) of the analyte is present. Therefore, the method may further be a method for characterizing aqueous solutions for the level of at least one analyte, wherein the sensor array comprises at least one poly-diacetylenes capable of a colorimetric response dependent on the level of said analyte. The level of an analyte may particularly be its concentration in the aqueous solutions in e.g. mM, g/mol, % (w/w) or % (V/V).

The aqueous solution for analysis by the method of the present invention may be a solution of importance in various fields and industries, such as food and beverage production, medicine including diagnostics, and environmental monitoring. Thus, in a preferred embodiment the aqueous solution is selected from the group consisting of beverage precursors, beverages, aqueous industrial waste, sewage, non-human biological samples, blood plasma, urine, and saliva.

More preferably, the aqueous solution is a beverage or precursor thereof. The beverage may be selected from the group consisting of beer, cider, white wine, rosé wine, red wine, dairy products, soft-drinks, alcopops and precursors thereof, most preferably beer and precursors thereof. Particularly the beverage precursor may be selected from the group consisting of wort and fermented wort.

The analytes of the present invention may be any organic molecules, ions or salts capable interacting with the PDA's of the sensor array. The organic molecule may preferably be an organic molecule with a molecular weight in the range of 5-2000 g/mol, such as 10-1500 g/mol, 20-1000 g/mol, such as preferably 30-500 g/mol. A preferred use of the present method is in the characterisation of liquid foodstuffs or beverages e.g. for human or animal consumption. Thus, in a preferred embodiment the at least one analyte, such as preferably all analytes are a flavour constituent of a beverage. One area where the present invention is envisioned to be particularly useful is in the characterisation of beer and/or beer precursors.

The flavour constituent present in beer may be selected from the group consisting of ethanol, carbonic acid, hop bitter substances (such as trans-isohumulone), hop oil constituents (such as myrcene, humulene, oxygenated humulenes), maltol, monosaccharides, disaccharides, banana esters (such as 3-methylbutyl acetate, 2-methylpropyl acetate), apple esters (such as ethyl hexanoate and ethyl octanoate), 3-methylbutanol, dimethyl sulfide, C₆-C₁₂ fatty acids (such as octanoic acid), acetic acid, propanoic acid, ethyl acetate, 2,3-butanedione, citric acid, maleic acid, polyphenols (such as leucocyanidin), trisaccharides (such as maltotriose), amino acids (such as proline), diacetyl acetylpropionyl, acetaldehyde, isobutylacetate, propanol, isobutanol, isoamylacetate, isoamylalcohol, ethyl caproate, ethyl caprylate, 2-phenylethylaceteate, caprylic acid, caproic acid, capric acid, linalool, limonene, pentanedione, A-decalactone, 2-phenylethanol, trans-2-noneal, 4-vinylguaiacol (4-VG), hydrogen sulfide, 3-methyl-2-butene-1-thiol, and sodium chloride. Particularly preferred flavour constituents include those selected from the group consisting ethanol, ethyl acetate, diacetyl, 4-vinylguaiacol, ethyl hexanoate, isoamylacetate, and acetylpropionyl.

In one embodiment the at least one analytes are one or more, preferably all of the compounds selected from the group consisting of ethanol, acetylpropionyl, ethyl acetate, 4-vinyl guaiacol, ethyl hexanoate, isoamylacetate, and diacetyl.

In one preferred embodiment the analyte of the present invention is ethanol, and the sensor array comprises at least one poly-diacetylenes capable of a colorimetric response dependent on the level of ethanol. The present inventors have found that the sensor array of the invention is capable of detecting and distinguishing between different levels of ethanol in an aqueous composition such as beer, but importantly they have also surprisingly found that the array is capable of simultaneously measuring the presence and level of other analytes, present in amounts much lower than ethanol. The level of ethanol in the aqueous solution may be in the range of 0.01-90% (V/V), such as 0.01-80% (V/V), 0.01-70% (V/V), 0.01-50% (V/V), 0.01-30% (V/V), 0.01-20% (V/V), 0.05-20% (V/V), 0.10-15% (V/V), 0.20-10% (V/V), such as preferably 0.5-8% (V/V). In one preferred embodiment the analyte of the present invention is acetylpropionyl, and the sensor array comprises at least one poly-diacetylenes capable of a colorimetric response dependent on the level of acetylpropionyl. The level of acetylpropionyl in the aqueous solution may be in the range of 0.01-90% (V/V), such as 0.01-80% (V/V), 0.01-70% (V/V), 0.01-50% (V/V), 0.01-30% (V/V), 0.01-20% (V/V), 0.05-20% (V/V), 0.10-15% (V/V), 0.20-10% (V/V), such as preferably 0.5-8% (V/V). In one preferred embodiment the analyte of the present invention is ethyl acetate, and the sensor array comprises at least one poly-diacetylenes capable of a colorimetric response dependent on the level of ethyl acetate. The level of ethyl acetate in the aqueous solution may be in the range of 0.01-90% (V/V), such as 0.01-80% (V/V), 0.01-70% (V/V), 0.01-50% (V/V), 0.01-30% (V/V), 0.01-20% (V/V), 0.05-20% (V/V), 0.10-15% (V/V), 0.20-10% (V/V), such as preferably 0.5-8% (V/V). In one preferred embodiment the analyte of the present invention is diacetyl, and the sensor array comprises at least one poly-diacetylenes capable of a colorimetric response dependent on the level of diacetyl. The level of diacetyl in the aqueous solution may be in the range of 0.01-90% (V/V), such as 0.01-80% (V/V), 0.01-70% (V/V), 0.01-50% (V/V), 0.01-30% (V/V), 0.01-20% (V/V), 0.05-20% (V/V), 0.10-15% (V/V), 0.20-10% (V/V), such as preferably 0.5-8% (V/V). In one preferred embodiment the analyte of the present invention is isoamyl alcohol, isobutanol, phenethyl alcohol, propanol or 4-VG, and the sensor array comprises at least one poly-diacetylenes capable of a colorimetric response dependent on the level of isoamyl alcohol, isobutanol, phenethyl alcohol, propanol or 4-VG. The level of isoamyl alcohol, isobutanol, phenethyl alcohol, propanol or 4-VG in the aqueous solution may be in the range of 0.01-90% (V/V), such as 0.01-80% (V/V), 0.01-70% (V/V), 0.01-50% (V/V), 0.01-30% (V/V), 0.01-20% (V/V), 0.05-20% (V/V), 0.10-15% (V/V), 0.20-10% (V/V), such as preferably 0.5-8% (V/V).

The sensor array used in the method of the present invention comprises spatially separated PDA polymers, which may be presented for the aqueous composition by various means. Hence, in one embodiment the at least two different poly(diacetylene) polymers are positioned in a vesicle or micelle. Alternatively, the at least two different poly(diacetylene) polymers are positioned on a solid support. The solid support may preferably selected from the group consisting of paper-based solid support, polymer based solid support, metal based solid support, inorganic porous material based solid support, electrospun fibres, carbon nanotube based solid support or any mixtures thereof, most preferably a paper-based solid support. Paper based solid supports have been found to be particularly useful for the present array, and provides for facile production of arrays comprising a plurality of PDA “spots”. The PDA spots may be positions on paper based solid support by various means, such as for example ink-jet printing of the monomers in solution.

The sensor array may comprise two or more different PDA polymers, which are spatially separated and individually addressable. More preferably the sensor array comprises at least 3 different poly(diacetylene) polymers, such as at least 4, at least 5, at least 10, such as at least 15 different poly(diacetylene) polymers. Even more preferably the sensor array comprises at least 3 different poly(diacetylene) polymers from Table 1, such as at least 4, at least 5, at least 10, such as at least 15 different poly(diacetylene) polymers selected from Table 1.

The present inventors have surprising found that the present method is capable of distinguishing between complex aqueous solutions comprising a plurality of analytes. Particularly it has been shown that the method may distinguish commercial beers, including commercial beers with the same ethanol percentage.

Thus, in a preferred embodiment the method of the present invention is capable of differentiating distinct beers or beer precursors. Even more preferably the method is capable of differentiating beers or precursors thereof from different batches and/or different breweries. Similarly, the methods can be used to test whether a test beers or wort can be considered to be similar to a reference beer or wort.

The present invention is useful for the characterisation and production monitoring of beer and precursors thereof. Thus, a further aspect of the present invention is a method for characterizing a beer or a beer precursor for multiple analytes, comprising the steps of
a) providing a sensor array comprising at least two different poly-diacetylenes, wherein said poly-diacetylenes are spatially separated and individually addressable,
b) contacting said sensor array with a sample of a beer or a beer precursor,
c) measuring the colorimetric response of said poly-diacetylenes to the beer or beer precursor, and
   wherein said poly-diacetylenes are polymers polymerised from a composition comprising a diacetylene monomer or mixtures thereof, and
   wherein said sensor array for each analyte comprises at least one poly-diacetylene capable of a colorimetric response to contact to said analyte, and wherein the analytes are flavour constituents of beer.

### Sensor Arrays of the Invention

Yet another aspect of the present invention relates to a sensor array comprising at least two different poly-diacetylenes,

wherein said poly-diacetylenes are spatially separated and individually addressable, and
wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, and
wherein said poly-diacetylenes are capable of a colorimetric response upon contact with an analyte.

Preferably the sensor array is as defined in the first aspect of the present invention. Thus, preferably the sensor array comprises the poly-diacetylenes are polymerised from diacetylene monomers as defined for the first aspect of the invention, or more preferably as defined according to formula (I) or (II) above. Also, the flavour constituents of beer are preferably as defined for the first aspect above. Generally, it should be noted that embodiments and features described in the context of one of the aspects of the present invention also apply to the other aspects of the invention.

The colorimetric responses as described for the present invention are as defined above a measurable colour change in the PDA as positioned on the sensor array. The colour change may be analysed e.g. by scanning the array (using e.g. a 600 DPI flatbed scanner) after subjecting it to the aqueous solution to be analysed and comparing to a scan of an analogous untreated array, or an array treated with a reference solution (e.g. water or a reference beer or wort). The colour change may be analysed according to the RGB changes (Red, Green Blue) by image software (e.g. imageJ®), to provide quantitative data. The measurements may preferably be performed in duplicates or triplicates to improve data reliability and statistical significance. The quantitative data may then be analysed using suitable statistical methods and software. Principal component analysis has proven particularly useful in this regards. For liquid based sensor arrays, e.g. based on miscelles, the colorimetric response is preferably determined or calculated from absorbance measurements.

The present invention is further defined by the following items:

1. A method for characterizing an aqueous solution for at least one analyte, comprising the steps of
a) providing a sensor array comprising at least two different poly-diacetylenes, wherein said poly-diacetylenes are spatially separated and individually addressable,
b) contacting said sensor array with a sample of said aqueous solution,
c) measuring the colorimetric response of said poly-diacetylenes to the aqueous solution,
   wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, wherein
   said poly-diacetylenes are capable of a colorimetric response upon contact with said analyte, and wherein
   the at least one analyte is selected from the group consisting of an organic molecule with a molecular weight below 2000 g/mol, salts thereof and an inorganic salt.

2. The method according to item 1, wherein the optionally substituted C₂-C₃₀ alkynyl comprises further diacetylene groups.

3. The method according to any one of items 1-2, wherein said one or more diacetylene monomer(s) is selected from the group of diacetylenes according to formula (I) or (II)

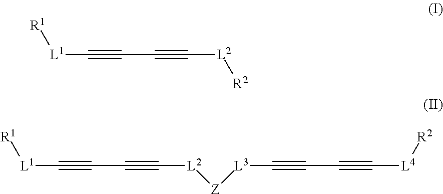

or mixtures thereof, wherein
L¹, L², L³ and L⁴ are the same or different and individually selected from the group consisting of an optionally substituted C₁-C₃₀ alkylene, an optionally substituted C₂-C₃₀ alkenylene, and an optionally substituted C₂-C₃₀ alkynylene,
R¹ and R² are the same or different and individually selected from the group consisting of —CH₃, OR³, SR³, —COOR³, —CONR⁴R⁵, wherein R³, R⁴, and R⁵ are individually selected from the group consisting of hydrogen, C₁-C₈ alkyl optionally substituted with a thiol, vinyl, or optionally substituted imidazolium, and a polyethylene glycol alkyl ether optionally substituted with a thiol, vinyl, or optionally substituted imidazolium, or are selected so that NR⁴R⁵ constitutes an amino acid,
Z is selected from the group consisting of optionally substituted alkylene, aryl, —CONH—(CH₂)x-HNCO— where X is an integer between 1 and 20, and heteroaryl.

4. The method according to item 3, wherein L¹, L², L³ and L⁴ are the same or different and individually selected from the group consisting of C₁-C₃₀, such as C₁-C₁₈, such as C₁-C₁₅, such as C₂-C₁₂ optionally substituted alkylene, alkenylene and alkynylene.

5. The method according to any one of items 3-4, wherein L¹, L², L³ and L⁴ are the same or different and individually selected from a —(CH₂)ₙ— group wherein n is 1-30, such as 1-20, 1-18, 1-15, such as preferably 1-12.

6. The method according to any one of items 3-5, wherein the carbon chain length of L¹ versus L² or L³ versus L⁴ may preferably be C₁-C₃₀ versus C₁₀-C₃₀, such as C₁-C₁₀ versus C₁₀-C₂₀, C₂-C₈ versus C₅-C₁₅, C₂-C₈ versus C₈-C₁₅, C₂-C₆ versus C₄-C₁₂, such as C₂-C₆ versus C₆-C₁₂.

7. The method according to any one of items 3-5, wherein at least one of L1 or L³ in the diacetylene according to formula (I) or (II) is different from at least one of L² and L⁴, and wherein at least one of L¹ or L³ is a C₁-C₁₅ optionally substituted alkylene, alkenylene or alkynylene, and at least one of L² and L⁴ is a C₁₆-C₃₀ optionally substituted alkylene, alkenylene or alkynylene.

8. The method according to any one of items 3-5, wherein at least one of L1 or L³ in the diacetylene according to formula (I) or (II) is different from at least one of L² and L⁴, and wherein at least one of L¹ or L³ is a C₁-C₁₀ optionally substituted alkylene, alkenylene or alkynylene, and at least one of L² and L⁴ is a C₁₁-C₂₀ optionally substituted alkylene, alkenylene or alkynylene.

9. The method according to any one of items 3-5, wherein at least one of L1 or L³ in the diacetylene according to formula (I) or (II) is different from at least one of L² and L⁴, and wherein at least one of L¹ or L³ is a C₂-C₈ optionally substituted alkylene, alkenylene or alkynylene, and at least one of L² and L⁴ is a C₉-C₁₅ optionally substituted alkylene, alkenylene or alkynylene.

10. The method according to any one of items 3-5, wherein at least one of L1 or L³ in the diacetylene according to formula (I) or (II) is different from at least one of L² and L⁴, and wherein at least one of L¹ or L³ is a C₅-C₈ optionally substituted alkylene, alkenylene or alkynylene, and at least one of L² and L⁴ is a C₉-C₁₂ optionally substituted alkylene, alkenylene or alkynylene.

11. The method according to any one of items 3-10, wherein R¹ and R² are the same or different and individually selected from the group consisting of —CH₃, and —COOR³.

12. The method according to any one of items 3-11, wherein R³, R⁴, and R⁵ are individually selected from the group consisting of hydrogen, and C₁-C₃ alkyl.

13. The method according to item 3, wherein L¹, L², L³ and L⁴ are the same or different and individually selected from a —(CH₂)ₙ— group wherein n is 1-20,

R¹ and R² are the same or different and individually selected from the group consisting of —CH₃, —COOR³, —CONR⁴R⁵, wherein R³, R⁴, and R⁵ are individually selected from the group consisting of hydrogen, and C₁-C₈ alkyl optionally substituted with a thiol, vinyl, or optionally substituted imidazolium, and a polyethylene glycol alkyl ether optionally substituted with a thiol, vinyl, or
optionally substituted imidazolium, or are selected so that NR⁴R⁵ constitutes an amino acid, and
Z is selected from the group consisting of optionally substituted alkylene, aryl, —CONH—(CH₂)x-HNCO— where X is an integer between 1 and 20, and heteroaryl.

14. The method according to any one of items 1-13, wherein the diacetylene monomers are selected from the group consisting of 5,7-hexadecadiynoic acid, 10,12-tricosadiynoic acid, and 10,12-pentacosadiynoic acid or mixtures thereof.

15. The method according to any one of items 3-13, wherein the amino acid is selected from the group consisting of Histidine, Isoleucine, Leucine, Lysine, Methionine, Phenylalanine, Threonine, Tryptophan, Valine, Arginine, Cysteine, Glutamine, Glycine, Proline, Serine, Tyrosine, Alanine, Asparagine, Aspartic acid, Glutamic acid.

16. The method according to item 15, wherein the amino acid is arginine.

17. The method according to any one of items 1-16, wherein at least one of the poly-diacetylenes is a polymer polymerized from a mixture comprising at least two different diacetylene monomers.

18. The method according to any one of items 1-17, wherein the concentration of diacetylene monomer or mixture thereof during polymerisation is in the range of 1-1000 mM, such as 2-500 mM, 5-200 mM, 8-150 mM, 10-100 such as preferably 20-75 mM.

19. The method according to any one of items 1-18, wherein the organic molecule is an organic molecule with a molecular weight in the range of 5-2000 g/mol, such as 10-1500 g/mol, 20-1000 g/mol, such as preferably 30-500 g/mol.

20. The method according to any one of items 1-19, wherein the method is a method for characterizing an aqueous solutions for at least a first analyte and a second analyte, and wherein at least one poly-diacetylene is capable of a colorimetric response upon contact with said first analyte and at least one poly-diacetylene is capable of a colorimetric response upon contact with said second analyte.

21. The method according to any one of items 1-19, wherein the method is a method for characterizing an aqueous solutions for multiple analytes, and wherein said sensor array for each analyte comprises at least one poly-diacetylene capable of a colorimetric response to contact to said analyte.

22. The method according to any one of items 1-21, wherein the at least one analyte, such as preferably all analytes are a flavour constituent of a beverage, preferably beer.

23. The method according to item 22, wherein the flavour constituent present in beer is selected from the group consisting of ethanol, carbonic acid, hop bitter substances (such as trans-isohumulone), hop oil constituents (such as myrcene, humulene, oxygenated humulenes), maltol, monosaccharides, disaccharides, banana esters (such as 3-methylbutyl acetate, 2-methylpropyl acetate), apple esters (such as ethyl hexanoate and ethyl octanoate), 3-methylbutanol, dimethyl sulfide, C₆-C₁₂ fatty acids (such as octanoic acid), acetic acid, propanoic acid, ethyl acetate, 2,3-butanedione, citric acid, maleic acid, polyphenols (such as leucocyanidin), trisaccharides (such as maltotriose), amino acids (such as proline), diacetyl acetylpropionyl, acetaldehyde, isobutylacetate, propanol, isobutanol, isoamylacetate, isoamylalcohol, ethyl caproate, ethyl caprylate, 2-phenylethylaceteate, caprylic acid, caproic acid, capric acid, linalool, limonene, pentanedione, A-decalactone, 2-phenylethanol, trans-2-noneal, 4-vinylguaiacol (4-VG), hydrogen sulfide, 3-methyl-2-butene-1-thiol, and sodium chloride.

24. The method according to item 23, wherein the at least one analyte is selected from the group consisting ethanol, ethyl acetate, diacetyl, 4-vinylguaiacol, ethyl hexanoate, isoamylacetate, and acetylpropionyl.

25. The method according to any one of items 1-24, wherein the method is a method for characterizing aqueous solutions for the level of at least one analyte, wherein the sensor array comprises at least one poly-diacetylenes capable of a colorimetric response dependent on the level of said analyte.

26. The method according to item 25, wherein the analyte is ethanol, and wherein the sensor array comprises at least one poly-diacetylenes capable of a colorimetric response dependent on the level of ethanol.

27. The method according to item 26, wherein the level of ethanol is in the range of 0.01-90% (V/V), such as 0.01-80% (V/V), 0.01-70% (V/V), 0.01-50% (V/V), 0.01-30% (V/V), 0.01-20% (V/V), 0.05-20% (V/V), 0.10-15% (V/V), 0.20-10% (V/V), such as preferably 0.5-8% (V/V).

28. The method according to any one of items 1-21, wherein the aqueous solution is selected from the group consisting of beverage precursors, beverages, aqueous industrial waste, sewage, non-human biological samples, blood plasma, urine, and saliva.

29. The method according to item 28, wherein the aqueous solution is a beverage or precursor thereof.

30. The method according to item 29, wherein the beverage is selected from the group consisting of beer, cider, white wine, rosé wine, red wine, dairy products, soft-drinks, alcopops and precursors thereof, most preferably beer.

31. The method according to any one of items 29-30, wherein the beverage precursor is selected from the group consisting of wort and fermented wort.

32. The method according to any one of items 1-31, wherein the at least two different poly(diacetylene) polymers are positioned in a vesicle or micelle.

33. The method according to any one of items 1-31, wherein the at least two different poly(diacetylene) polymers are positioned on a solid support.

34. The method according to any one of items 1-33, wherein there are at least 3 different spatially separated poly(diacetylene) polymers, such as at least 4, at least 5, at least 10, such as at least 15 different poly(diacetylene) polymers.

35. The method according to any one of items 33-34, wherein the solid support is selected from the group consisting of paper-based solid support, polymer based solid support, metal based solid support, inorganic porous material based solid support, electrospun fibres, carbon nanotube based solid support or any mixtures thereof.

36. The method according to any one of items 1-35, wherein said method is capable of differentiating distinct beers or beer precursors.

37. The method according to item 36, wherein said method is capable of differentiating beers or precursors thereof from different batches.

38. The method according to item 36, wherein said method is capable of differentiating beers or precursors thereof from different breweries.

39. A method for characterizing a beer or a beer precursor for multiple analytes, comprising the steps of
a) providing a sensor array comprising at least two different poly-diacetylenes, wherein said poly-diacetylenes are spatially separated and individually addressable,
b) contacting said sensor array with a sample of a beer or a beer precursor,
c) measuring the colorimetric response of said poly-diacetylenes to the beer or beer precursor, and
   wherein said poly-diacetylenes are polymers polymerised from a composition comprising a diacetylene monomer or mixtures thereof, and
   wherein said sensor array for each analyte comprises at least one poly-diacetylene capable of a colorimetric response to contact to said analyte, and
   wherein the analytes are flavour constituents of beer.

40. The method according to item 39, wherein the poly-diacetylenes are polymerised from diacetylene monomers according to any of items 1-18.

41. The method according to any one of items 40-41, wherein the flavour constituents of beer are as defined in any one of items 23-24.

42. A method for comparing a test aqueous solution with a reference aqueous solution comprising at least one analyte, comprising the steps of
a) providing at least two identical sensor arrays comprising at least two different poly-diacetylenes, wherein said poly-diacetylenes are spatially separated and individually addressable,
b) contacting a first sensor array with a sample of the test aqueous solution and a second sensor array with a reference aqueous solution,
c) comparing the colorimetric response of said poly-diacetylenes of the first sensor array to the colorimetric response of said poly-diacetylenes of the second sensor array,
   wherein a similar colorimetric response of the first sensor array and the second sensor array indicates that the test aqueous solution is similar to the reference aqueous solution; and
   wherein said poly-diacetylenes are polymers polymerised from a composition comprising a diacetylene monomer or mixtures thereof.

43. The method according to item 42, wherein the poly-diacetylenes are as defined in any one of items 1-18 or 32-34.

44. The method according to any one of items 42-43, wherein the analyte is as defined in any one of items 1, 19, or 22-27.

45. The method according to any one of items 42-44, wherein the aqueous solution is as defined in any one of items 28-31.

46. The method according to any one of items 42-45, wherein multiple reference aqueous solutions and/or multiple test aqueous solutions are compared.

47. The method according to any one of items 42-46, wherein a similar colorimetric response is a colorimetric response value within 10% for each sensor, such as within 8%, such as 6%, 4%, 3%, 2%, 1%, 0.5%, such as preferably 0.1% for each sensor.

48. A sensor array comprising at least two different poly-diacetylenes, wherein said poly-diacetylenes are spatially separated and individually addressable, and

wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, and
wherein said poly-diacetylenes are capable of a colorimetric response upon contact with an analyte.

49. The sensor array according to item 48, wherein the poly-diacetylenes are as defined in any one of items 1 to 18 or 32-34.

50. The sensor array according to any one of items 48-49, wherein the array comprises at least one poly-diacetylene polymerised from a diacetylene monomer, wherein R¹ and R² are the same or different and individually selected from the group consisting of —CH₃, OR³, SR³, —COOR³, —CONR⁴R⁵,

wherein R³, R⁴, and R⁵ are individually selected from the group consisting of C₁-C₈ alkyl substituted with a thiol, vinyl, or optionally substituted imidazolium, and a polyethylene glycol alkyl ether optionally substituted with a thiol, vinyl, or optionally substituted imidazolium, or are selected so that NR⁴R⁵ constitutes an amino acid.

All patent and non-patent references cited in the present application, are hereby incorporated by reference in their entirety.

The invention will now be described in further details in the following non-limiting examples.

### EXAMPLES

### Materials and Methods

### Di-Acetylene Monomers:

10,12-tricosadiynoic acid (98%, T),
10,12-pentacosadiynoic acid (97%, P),
5,7-hexadecadiynoic acid (97%, H),

Further monomers tested are in accordance with Table 2:

TABLE 2
tested DA monomers (No. 1-3 being H, T, P above)

| No | Chemical Structure | MW |
|---|---|---|
| 1 | 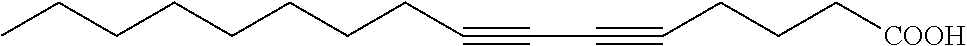 [SMILES: CCCCCCCCC#CC#CCCCC(=O)O] | 248.36 |
| 2 | 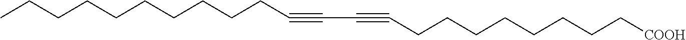 [SMILES: CCCCCCCCCCC#CC#CCCCCCCCCC(=O)O] | 346.55 |
| 3 | 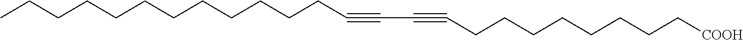 [SMILES: CCCCCCCCCCCCC#CC#CCCCCCCCCC(=O)O] | 374.60 |
| 4 | 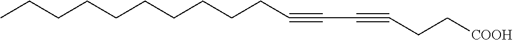 [SMILES: CCCCCCCCCCC#CC#CCCC(=O)O] | 262.39 |
| 5 | 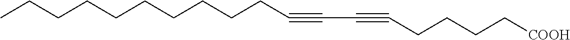 [SMILES: CCCCCCCCCCC#CC#CCCCCC(=O)O] | 290.45 |
| 6 | 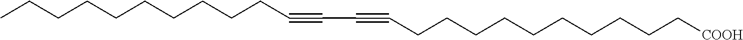 [SMILES: CCCCCCCCCCC#CC#CCCCCCCCCCCC(=O)O] | 374.61 |
| 7 | 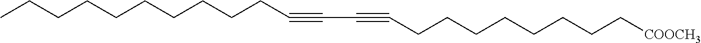 [SMILES: CCCCCCCCCCC#CC#CCCCCCCCCC(=O)OC] | 360.58 |
| 8 | 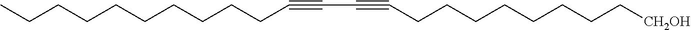 [SMILES: CCCCCCCCCCC#CC#CCCCCCCCCCO] | 332.57 |
| 9 | 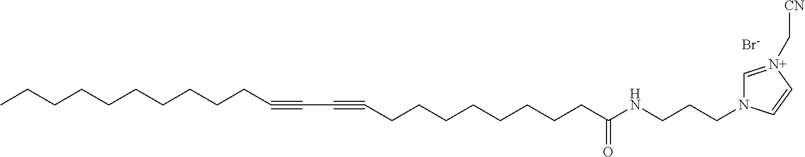 [SMILES: CCCCCCCCCCC#CC#CCCCCCCCCC(=O)NCCCn1cc[n+](CC#N)c1 ; [Br-]] | 573.66 |
| 10 | 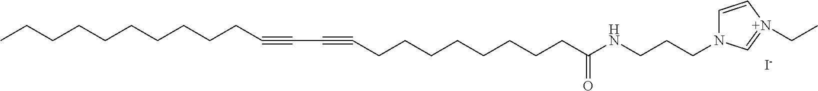 [SMILES: CCCCCCCCCCC#CC#CCCCCCCCCC(=O)NCCCn1cc[n+](CC)c1 ; [I-]] | 609.68 |
| 11 | 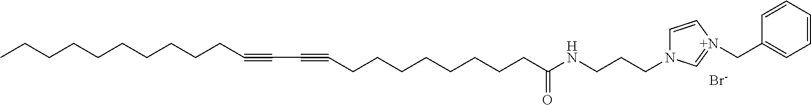 [SMILES: CCCCCCCCCCC#CC#CCCCCCCCCC(=O)NCCCn1cc[n+](Cc2ccccc2)c1 ; [Br-]] | 624.75 |
| 12 | 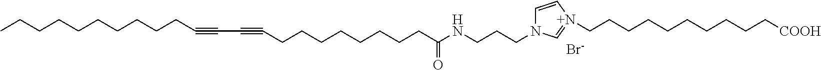 [SMILES: CCCCCCCCCCC#CC#CCCCCCCCCC(=O)NCCCn1cc[n+](CCCCCCCCCCC(=O)O)c1 ; [Br-]] | 718.91 |
| 13 | 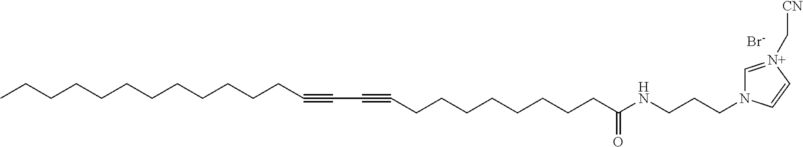 [SMILES: CCCCCCCCCCCCC#CC#CCCCCCCCCC(=O)NCCCn1cc[n+](CC#N)c1 ; [Br-]] | 601.72 |
| 14 | 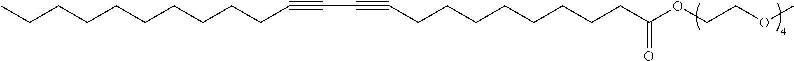 | 536.79 |
| 15 | 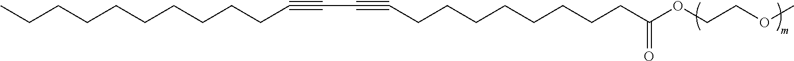 | 894.55 |
| 16 | 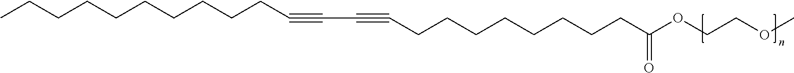 | 5329.18 |
| 17 | 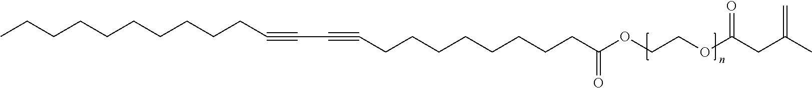 | 705.55 |
| 18 | 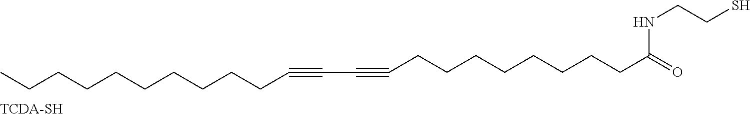 | 405.69 |
| 19 | 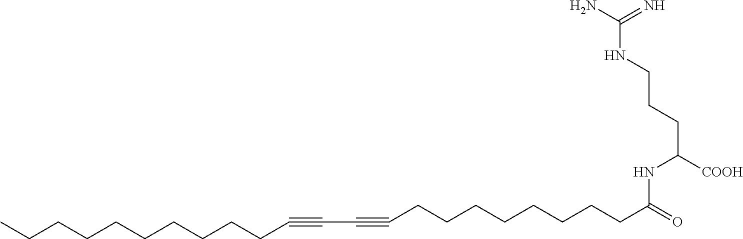 [SMILES: CCCCCCCCCCC#CC#CCCCCCCCCC(=O)NC(CCCNC(=N)N)C(=O)O] | 502.74 |
| 20 | 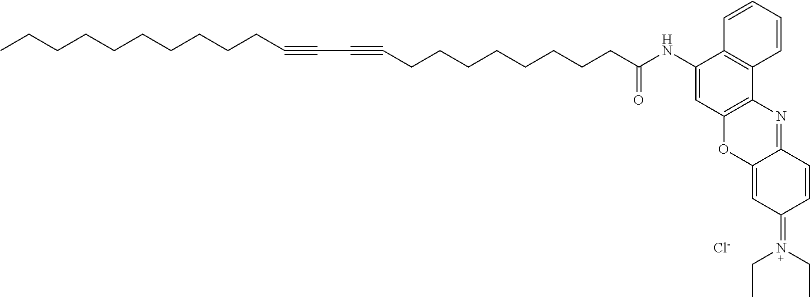 | 682.39 |
| 21 | 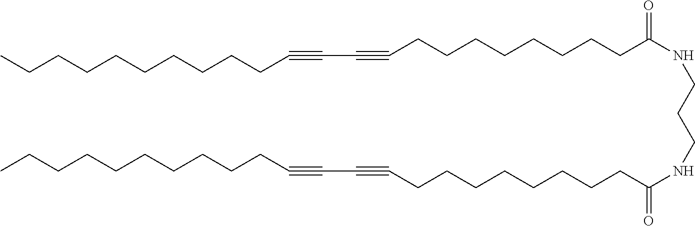 [SMILES: CCCCCCCCCCC#CC#CCCCCCCCCC(=O)NCCCNC(=O)CCCCCCCCC#CC#CCCCCCCCCCC] | 731.21 |

### Test Analytes

ethyl acetate (EA),

diacetyl (DAc)

acetylpropionyl (AP)

4-vinyl guaiacol (4-VG)

Further esters and alcohol analytes were tested as apparent from the examples.

### Test Beers (DK Commercial Names)

Carlsberg Nordic (CB N, 0.5% (V/V) ethanol)
Carlsberg Classic (CB C, 4.6% (V/V) ethanol)
Wiibroe Årgangsøl (WB, 10.6% (V/V) ethanol)
Tuborg Classic (TB C, 4.5% (V/V) ethanol)

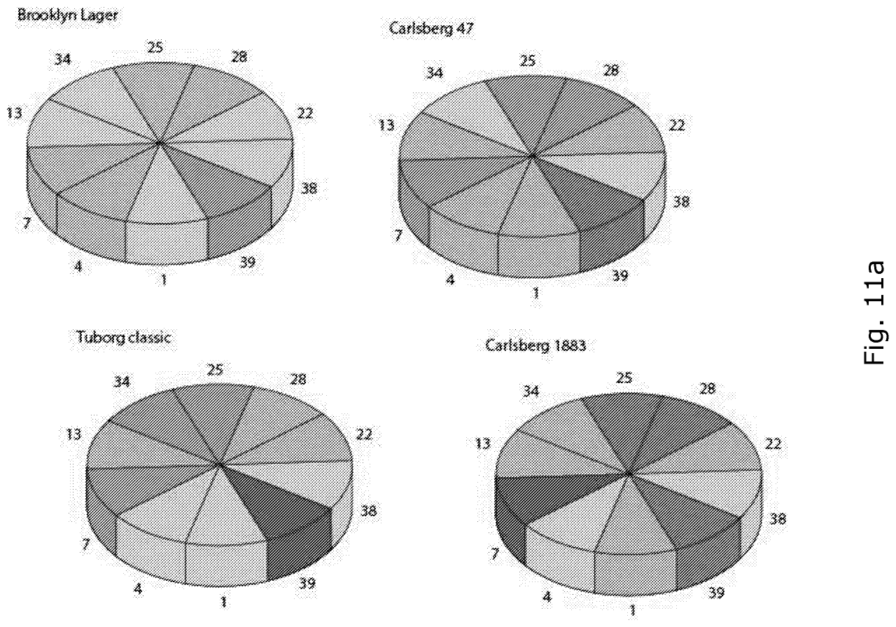

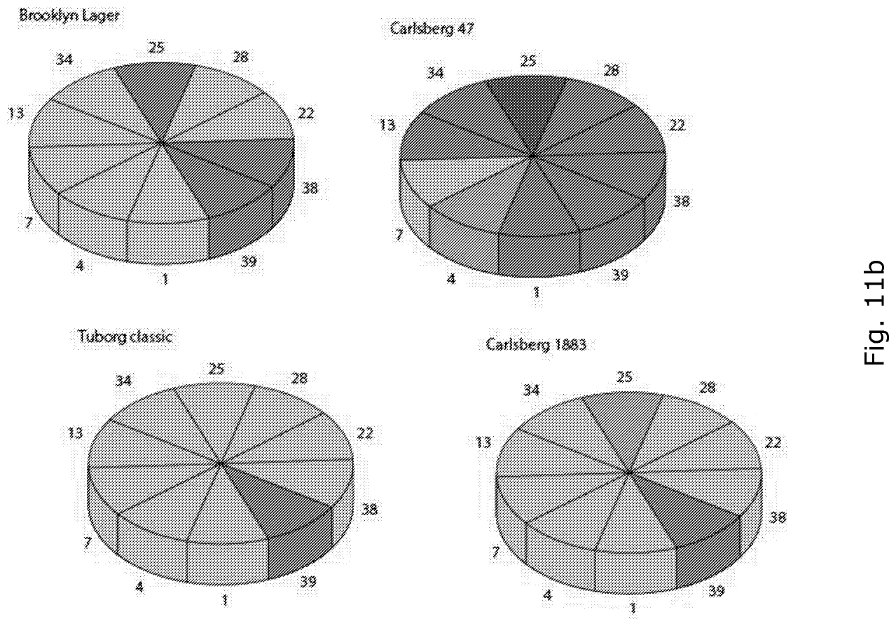

Further commercial beers were tested in accordance with FIG. 11a-b.

Diacetylene monomers (DA's) and test analytes were purchased from Sigma-Aldrich or synthesized as in example 12 or via literature methods for certain DA's. Beers were purchased from retail providers.

Prior to use for the sensor fabrication, the diacetylene acids were purified, using an adapted published protocol (M. Roman and M. Baranska, spectrochim. Acta Mol. Biomol. Spectrosc., 2015, 127, 652.), by dissolving 200 mg diacetylene acid (DA) in chloroform, filtering and retrieving of the diacetylene acids from the filtrate by evaporation of the chloroform overnight. The purification was conducted in the dark to prevent unwanted polymerisation of the monomers.

Ethanol (EtOH), chloroform, and filter paper (qualitative filter paper 600, medium filtration rate particle retention 10-20 μm) were obtained from VWR.

### Sensor Preparation

100 mM DA stock solutions were prepared in chloroform and used to prepare the different mixed solutions as well as dilution series (75, 50, 20 and 10 mM solution). The paper-based PDA sensors were fabricated by placing desired DA solution dropwise in rows on filter paper using a small glass capillary, which was cleaned with chloroform between the use of different solutions. The samples were placed in the fume hood to dry for 1 h at room temperature followed by UV crosslinking (6 W, λ=254 nm) for 1 min. Solution phase vesicle sensors were prepared in accordance with example 9 and FIG. 9.

### Analytes

0-15% EtOH in double distilled water mixtures were prepared. The 5% EtOH solution was supplemented with 0.1-10 mM (2-185 ppm) EA, 0.1-9 mM (2-155 ppm) DAc, or 0.1-10 mM (2-186 ppm) AP. The solutions were freshly prepared before usage. Four different beers Carlsberg Nordic: (CB N, 0.5% EtOH), Carlsberg Classic (CB C, 4.6% EtOH) Wiibroe Årgangsøl (WB, 10.6% EtOH) and Tuborg Classic (TB C, 4.6% EtOH) were purchased in normal retail. Prior to exposure to the sensors, the cans were opened and let to degas for 30 min.

### Detection

The paper-based PDA sensors were exposed to the different analyte solutions by covering the PDA spot with analyte solution, left to incubate for 10 s followed by drying for 1 h at room temperature. The paper was scanned (600 dpi scanner) to analyse the RGB changes in comparison to an untreated PDA sensor using the Image software (ImageJ®). The desired data set of RGB numerical data, which may be represented by red chromaticity shift (RCS) values or changes in Hue values, were tabulated and used directly or optionally analysed by principal component (PC) analysis (Excel PCA plug-in software [XLSTAT version 2018.2]) to identify data clusters in PCA score plots. Each experiment was done in three independent repeats in duplicates.

A change in Hue value (ΔHue) is calculated as follows: RGB Intensity values are converted to 0<I<1 by dividing each by 255 (for 8-bit color depth).

$$r = \frac{R}{255} g = \frac{G}{255} b = \frac{B}{255}$$

We find the maximum and minimum from r, g, b

If r is max:

$$Hue = 60 * ( \frac{g - b}{max - min} )$$

If g is max:

$$Hue = 60 * ( 2.0 + \frac{b - r}{max - min} )$$

If b is max:

$$Hue = 60 * ( 4.0 + \frac{r - g}{max - min} )$$

(If Hue<0 then Hue=Hue+360)

Finally

ΔHue=Hue_{after}−Hue_{be fore}

Red chromacity shift (RCS) is calculated as follows:

First Red chromaticity (RC) level is calculated as:

$$r = \frac{R}{R + G + B}$$

RCS is then calculated as:

$$RCS = \frac{{r}_{sample} - {r}_{0}}{{r}_{max} - {r}_{0}} * 1 \; 00 \%$$

### Where

rₛₐₘₚₗₑ is the intensity reading after exposure to the sample.
r₀ is the intensity reading before exposure to the sample.
rₘₐₓ intensity reading after exposure to 100% EtOH as positive control for max red shift.

Solution phase sensors were monitored using absorbance measurements (PerkinElmer EnSight™ Multimode Plate Reader), and analysed by calculating a colour specific colorimetric response (CR_{[colour]}) values of the sensors before and after interaction with the analyte solutions. The value of CR_{blue} indicates how big the blue colour differences are, which indicates how sensitive the sensors are to the analyte solutions. CR_{blue} is calculated as following:

$$CR ( blue ) = \frac{( P {B}_{b -} P {B}_{a} )}{P {B}_{b}} \times 100$$

Here PB_{b} (Percent of Blue) and PBₐ are the blue percentages of the sensors before and after interaction with analyte solutions, respectively, where:

$$P B = \frac{{A}_{640}}{{A}_{640} + {A}_{548}} \times 100$$

Here A640 is the absorbance at 640 nm, which represents the blue colour of the system, and A548 is the absorbance at 548 nm, which represents the red colour of the system.

### Example 1—Sensor Stability when Exposed to Ambient Environment

Paper-based PDA sensors were fabricated from 1 mM H, P and T as well as their mixtures (H/T, P/H, and T/P, all 1:1 vol-ratio). With the aim to characterize the stability of the paper-based PDA sensors i.e., their tendency to change colour in the absence of any specific stimuli, they were exposed to the environment between 2 and 1440 min and the RGB changes were compared to the sensor at time zero. The scanned images of the sensor arrays visually showed a red colour shift for all H containing sensors, while the others preserved their original blue colour.

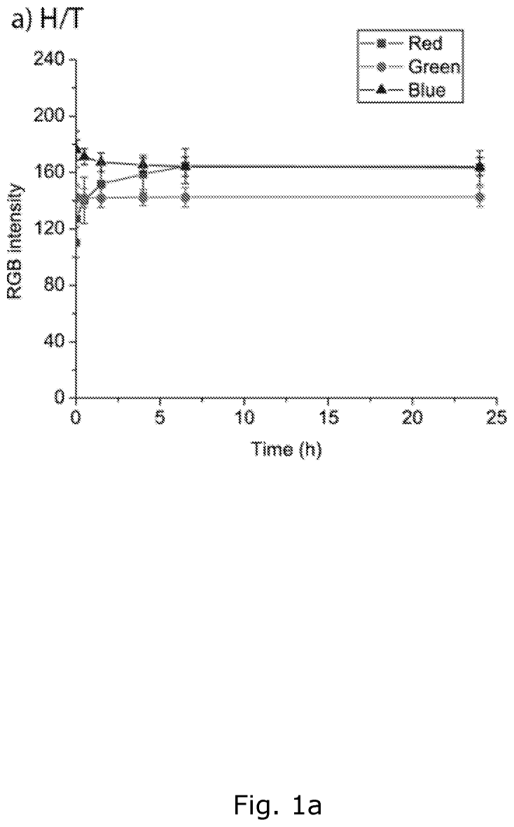

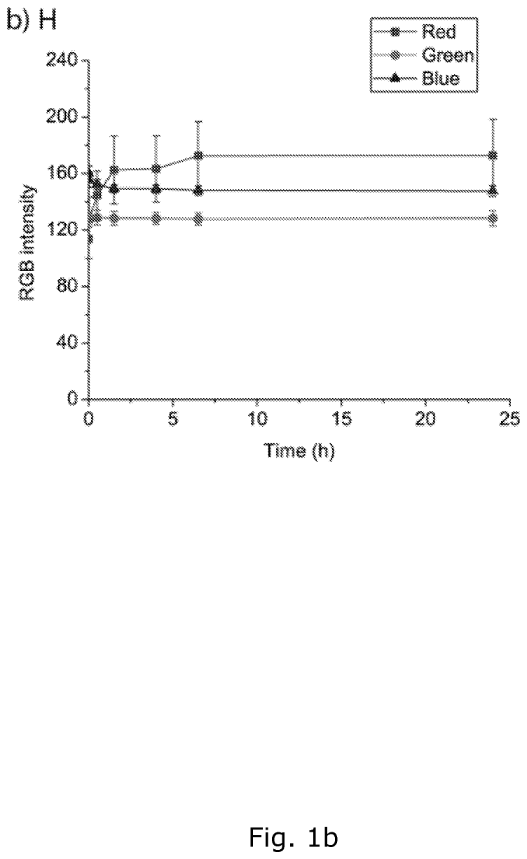

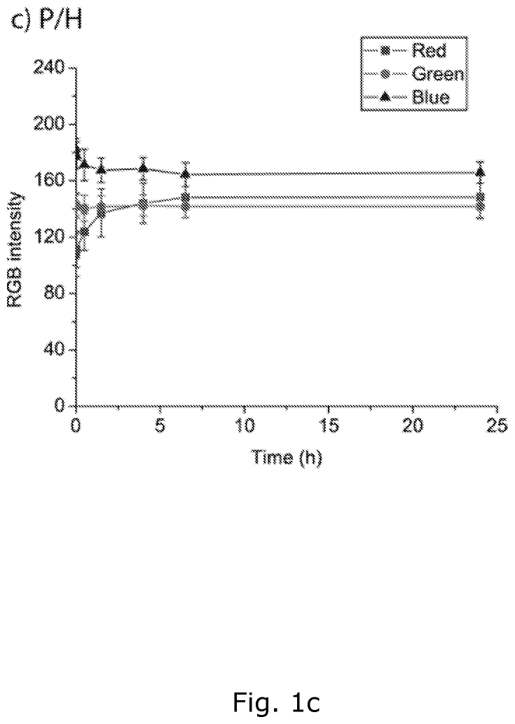

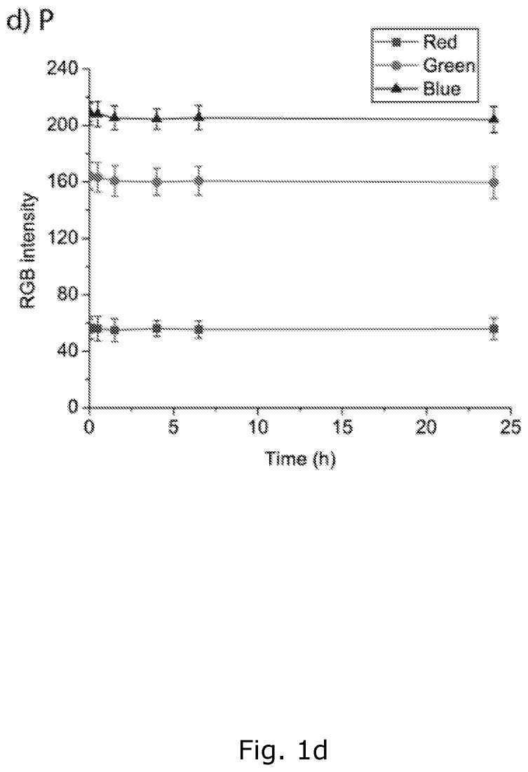

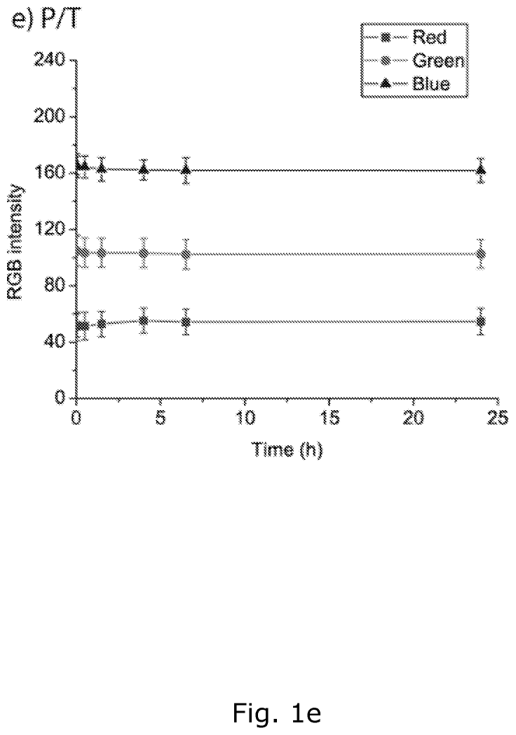

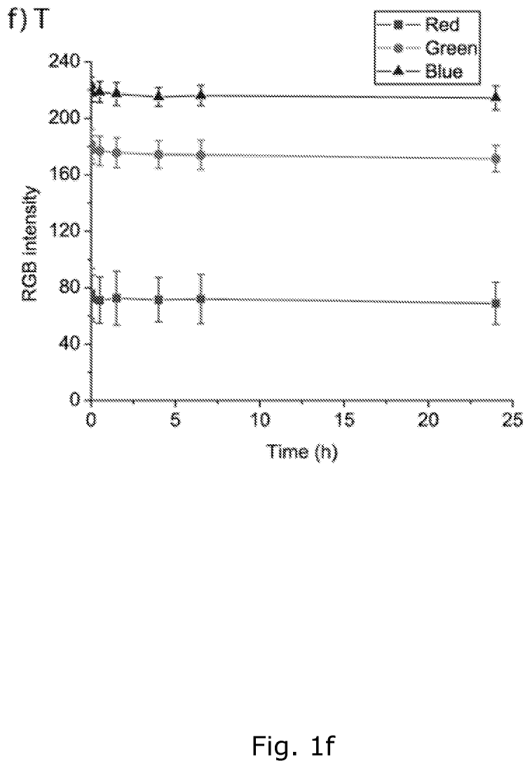

The specific RGB intensity plots confirmed this observation (FIGS. 1a-f). These plots also illustrated that the H-containing sensors stabilized after about 7 h. Therefore, for all the subsequent experiments, the paper-based PDA sensors were used about 12 h after fabrication.

### Example 2—Mapping Water-Ethanol Mixtures: Da Monomer Ratios

The first step towards the use of the paper-based PDA sensor in the context of alcoholic beverages, the effect of ethanol (EtOH) in water on the RGB colour change has to be considered.

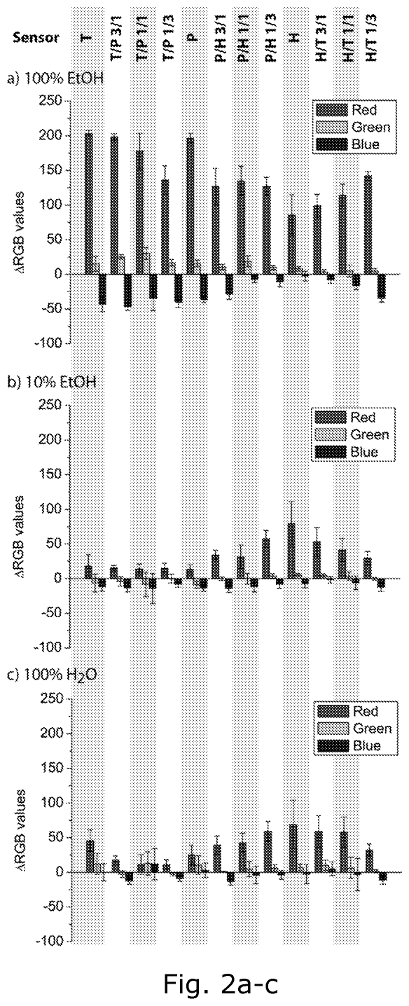

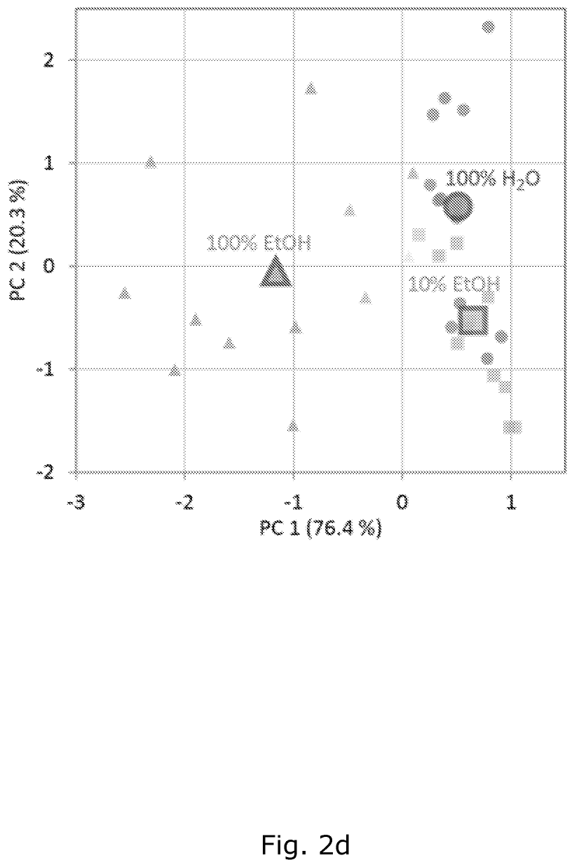

Different paper-based PDA sensors were fabricated from 1 mM H, P and T as well as their mixtures (H/T, P/H, and T/P, 3:1, 1:1 and 1:3 vol-ratio). The RGB colour change of these arrays before and after exposure to 100% EtOH, 10% EtOH and 100% ultrapure water (H₂O) was assessed. The scanned images of the sensors showed a strong red colour shift for 100% EtOH in all cases. Further, only H containing sensors exhibited a visible colour change upon exposure to 10% EtOH and 100% H₂O. The specific RGB intensity plots confirmed that 100% EtOH led to the largest changes in red and blue for all tested sensors while changes in H containing sensors were dominant for exposure to 10% EtOH and 100% H₂O (FIGS. 2a-c).

A statistical multivariate analysis was required for efficient pattern recognition and comparison. Therefore, principal component analysis (PCA) was used to generate coordinates represented by the PCs from the given set of colorimetric data. When comparting the sensor responses to 100% EtOH, 10% EtOH and 100% H₂O, the PC score plots showed that the first and second components (PC1 and PC2) accounted for 96.7% of the total variance (FIG. 2d). Further, the small symbols represent the individual sensor averages while the large symbols denote the group means. Three discrete clusters corresponding to the three tested solutions could be identified in this 2D plot, illustrating that the PDA sensor array could discriminate e.g. the 10% EtOH from the 100% H₂O solution.

### Example 3—Mapping Water-Ethanol Mixtures: Da Monomer Concentrations

Following on, to further improve the sensitivity and selectivity of sensor arrays assembled from T and P, the DA concentration used for their fabrication was stepwise lowered from 100 mM to 10 mM. We hypothesize that lower amounts of PDA on the paper might exhibit a more sensitive response upon exposure to different solutions. Since the response from sensors consisting (partly) of H showed visible blue-to-red colour shifts when 100 mM DAs were used during the fabrication, no lower concentrations were tested for this component.

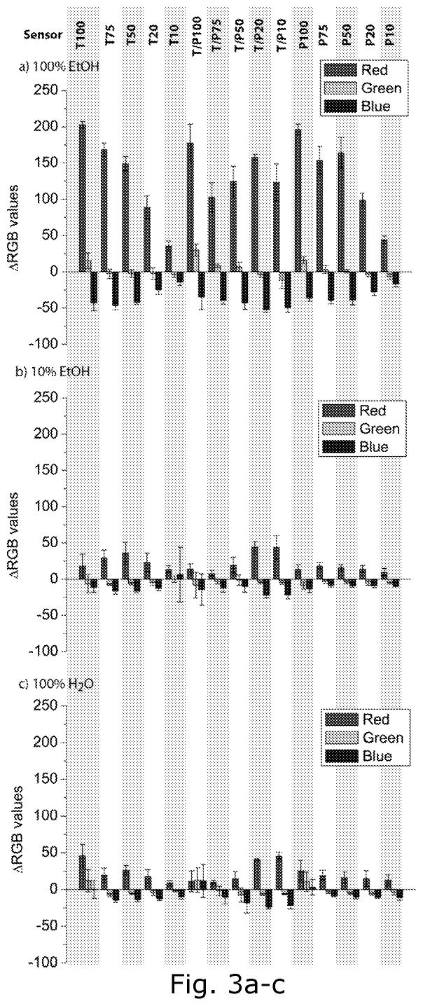

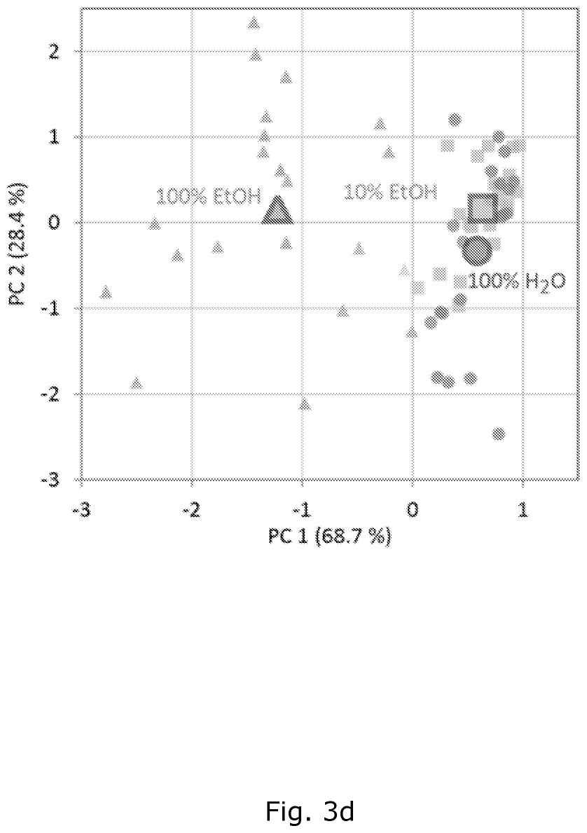

Scanned images of sensor arrays fabricated from different concentrations of T, T/P (1:1 vol-ratio) and P before and after exposure to 100% EtOH, 10% EtOH and 100% H₂O revealed a visual blue-to-red shift which varied for different DA concentrations. The specific RGB intensity plots supported this qualitative assessment (FIGS. 3a-c). Specifically, while P sensors seemed to provide the same response independent on the used DA concentration during fabrication, lowering the DA concentration in T/P sensors yielded in larger colour shifts. Further, T sensors showed an analyte specific response i.e., T50 and T100 led to the largest colour change for 10% EtOH and 100% H₂O, respectively.

The PCA, with PC1 and PC2 accounted for 97.1% of the total variance, revealed a very distinct cluster for sensors exposed to 100% EtOH as well as the possibility to discriminate between 10% EtOH and 100% H₂O (FIG. 3d).

### Example 4—Mapping Model Flavours: Ethanol

In a next step, the most promising sensor arrays were employed to assess the possibility to distinguish between aqueous solutions containing between 2.5 and 15% EtOH (2.5, 5, 10, 15% EtOH solutions).

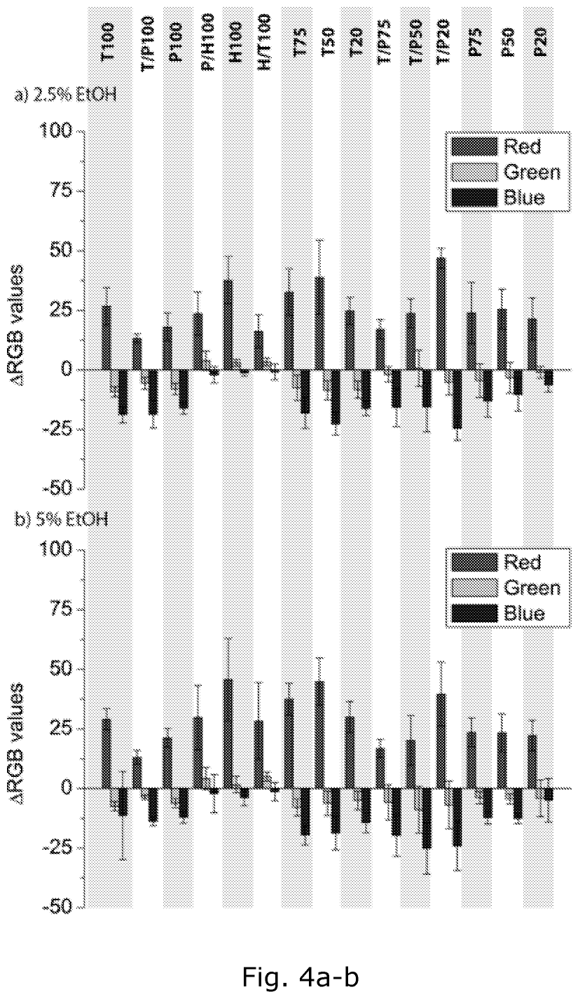

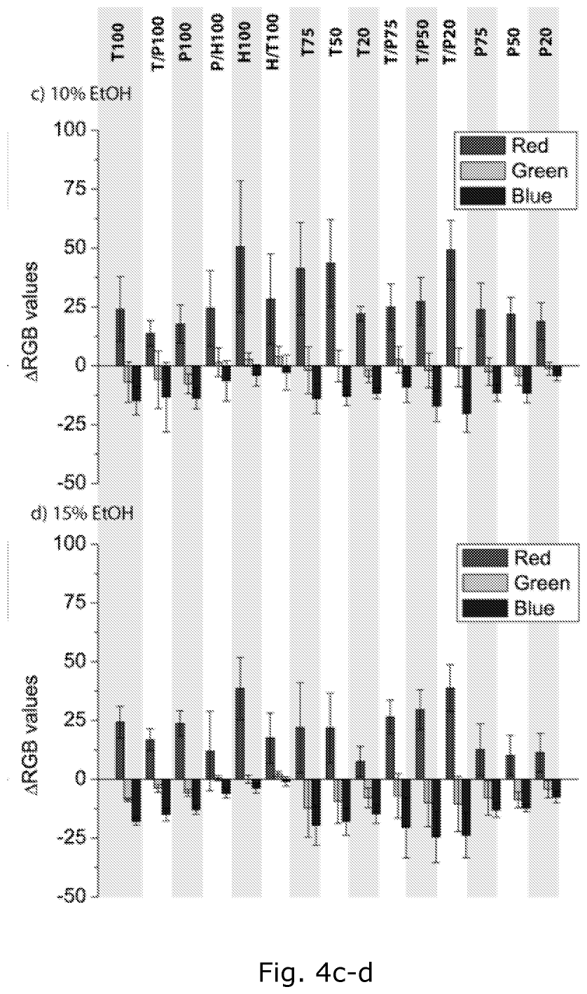

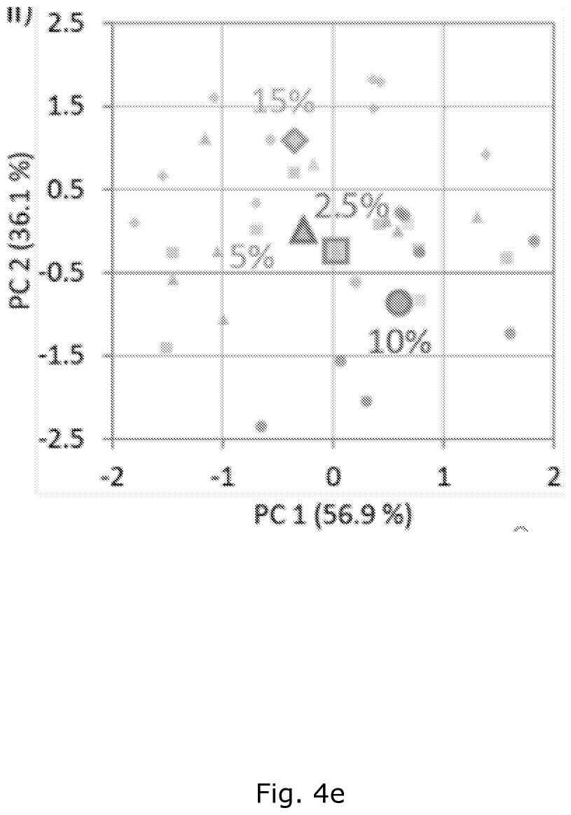

Sensor arrays fabricated from pure 100 mM H, T, and P or from a 1:1 vol-ratio between these different DAs, as well as different concentrations of T, T/P (1:1 vol-ratio) and P showed a visible response after exposure to 2.5, 5, 10, 15% EtOH solutions. In the former case, the red-shift was best visible for H containing sensors. Additionally, visible changes in the sensor colours could be observed for almost all sensors fabricated from lower DAs concentrations (20, 50, 75 mM). The specific RGB intensity plots supported this qualitative assessment illustrates that (FIGS. 4a-d).

The PCA revealed a very distinct cluster for sensors exposed to 100% EtOH as well as the possibility to discriminate between 2.5, 5, 10, 15% EtOH solutions in H₂O (FIG. 4e).

### Example 5—Mapping Model Flavours: Ethyl Acetate

With the aim to increase the complexity of the analyte solution, 5% EtOH solutions were supplemented with pure compounds with relevance in the flavour analysis of beer, starting with ethyl acetate.

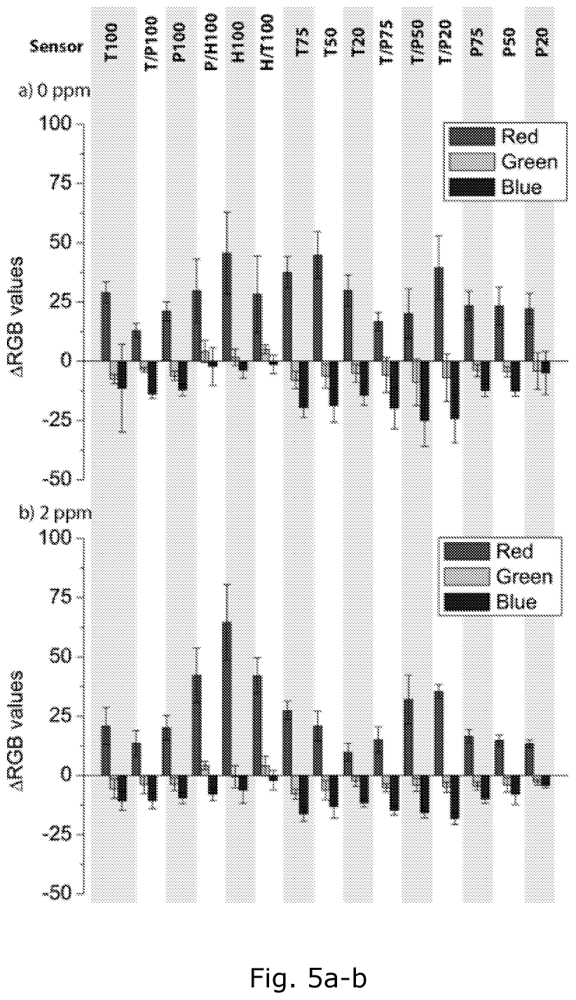

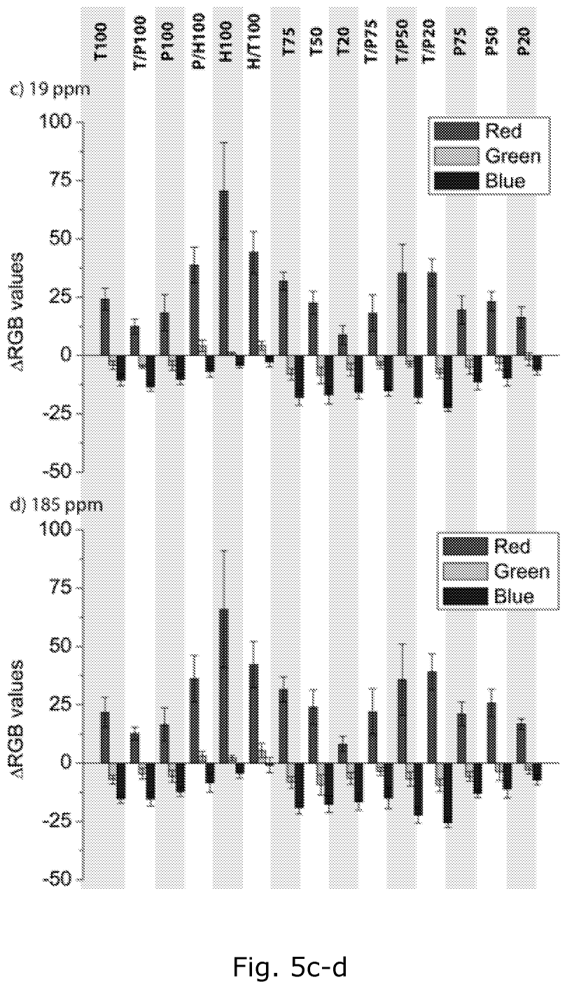

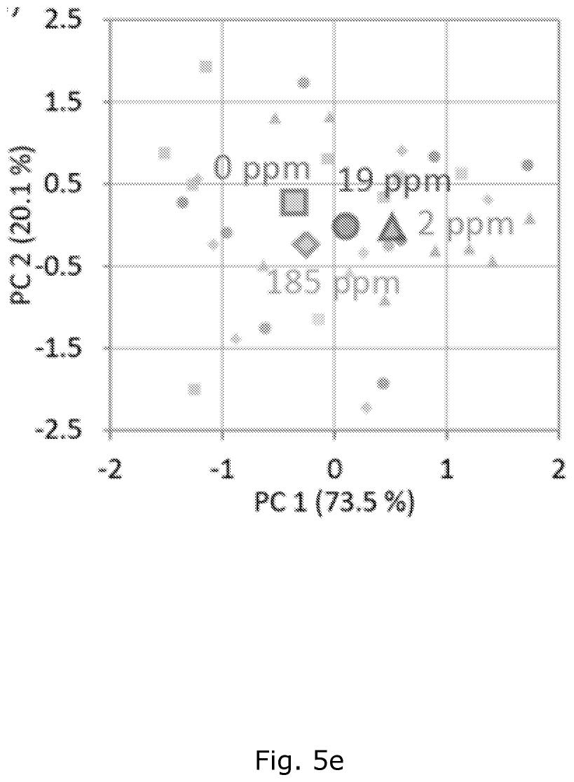

Ethyl acetate (EA) is a naturally occurring during yeast fermentation, but it can become an off-flavour in beer when present at too high levels. Different amounts of EA (0-185 ppm) were added to a 5% EtOH solution to test the sensitivity of different sensor for this compound. First, although the scanned images of the T, P and H as well as their 1:1 mixtures (100 mM) did not show any visible differences in the blue-to-red shift, the specific RGB intensity plots revealed that the presence of EA could be detected down to 2 ppm (FIG. 5a-d).

When sensors consisting of T, T/P and P with lower DA concentrations were used (20, 50, 75 mM), the sensor responses varied for different amounts of EA supplemented to the 5% EtOH solutions. Although the differences were barely visible in the scanned images of the sensors, they could be identified in the specific RGB intensity plots (FIG. 5a-d) and the PC analysis (FIG. 5e).

### Example 6—Mapping Model Flavours: Diacetyl

Diacetyl (DAc) is a yeast product formed in the early stage of the fermentation cycle, responsible for butte or butter scotch flavour in beers. While it is desired for certain types of brews, DAc is often also considered a rancid off-flavour.

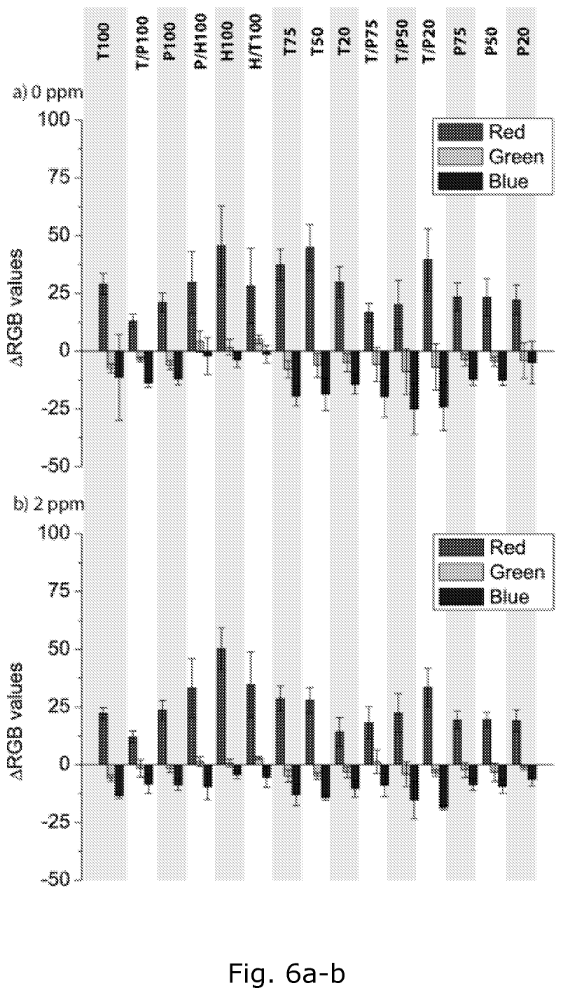

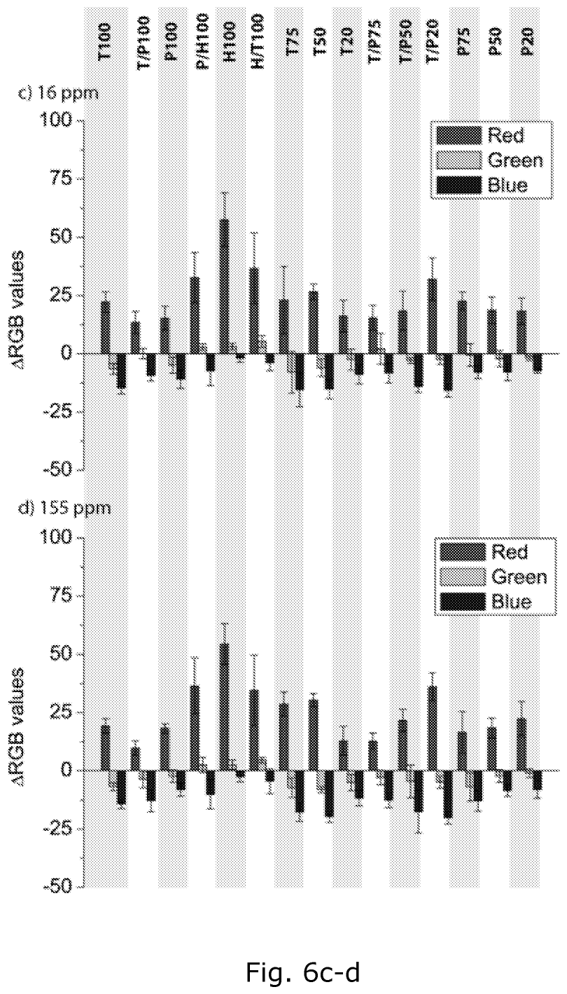

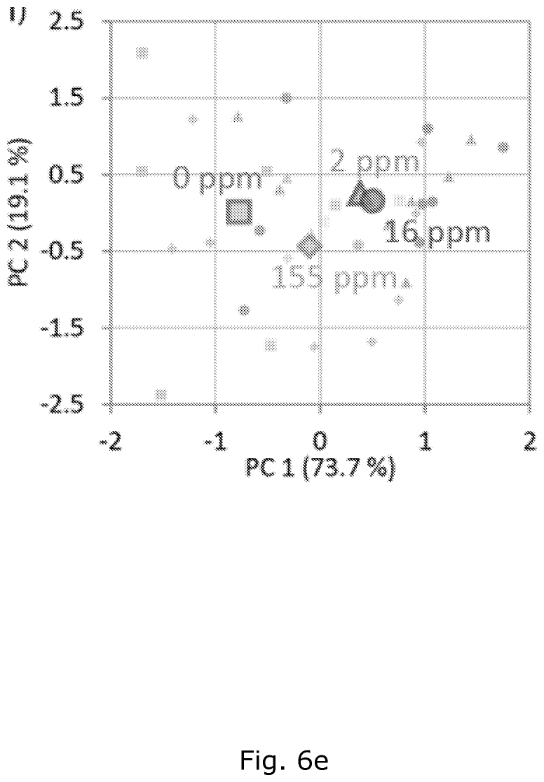

A 5% EtOH solution was supplemented with different amounts of DAc (0-866 ppm) to assess the sensitivity of the sensors towards this specific molecule. When using sensors fabricated from T, P and H as well as their 1:1 mixtures (100 mM), the presence of DAc in the EtOH solution was difficult to detect in the specific RGB intensity plots (FIGS. 6a-d). However, sensors consisting of T, T/P and P with lower DA concentrations (20, 50 or 75 mM) could readily identify the supplemented DAc. While the differences were difficult to observe in the scanned images and the specific RGB intensity plots, they became obvious in the PC analysis (FIG. 6e). Interestingly, the pure 5% EtOH solution could clearly be separated from the DAc supplemented solutions. However, when the DAc concentration became too high, distinction became less effective with these sensors. Specifically, 5% EtOH solutions with 155 ppm DAc could be separated from solutions supplemented with 2 or 16 ppm.

### Example 7—Mapping Model Flavours: Acetylpropionyl

Acetylpropionyl (AP) formed during fermentation gives a honey-like flavour to beverages.

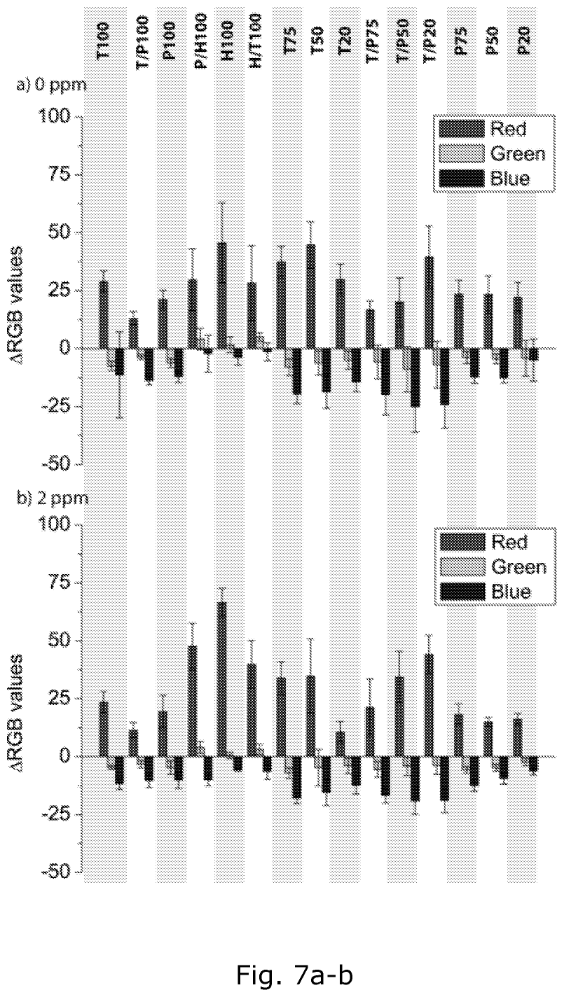

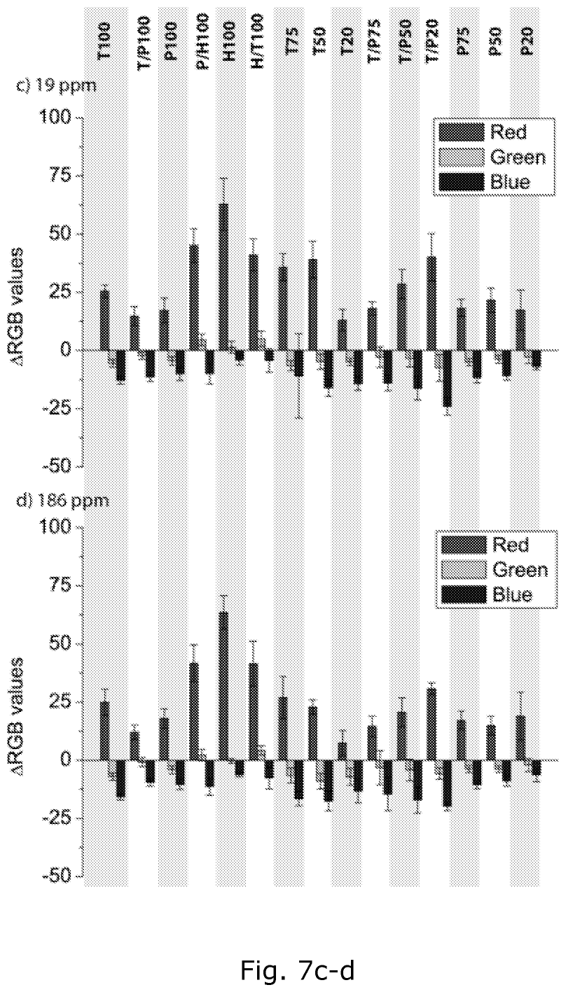

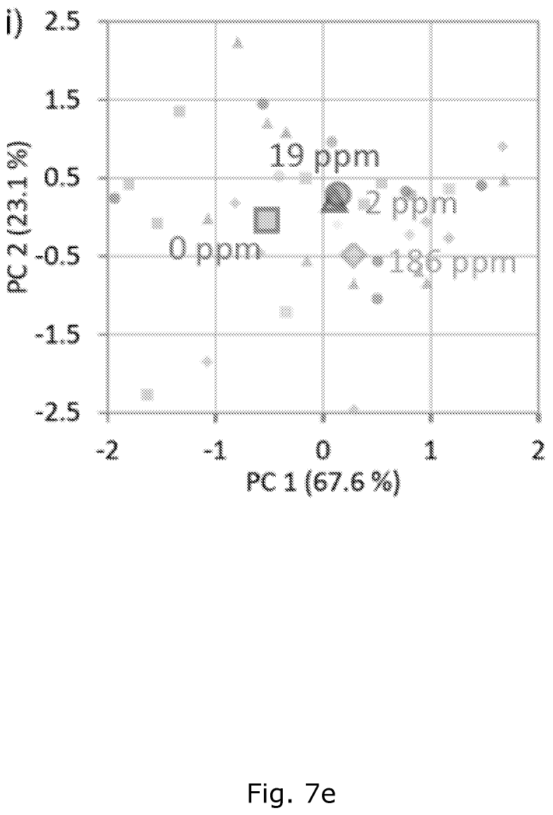

A 5% EtOH solution was supplemented with different amounts of AP (0-1000 ppm) to assess the sensitivity of the sensors towards this particular compound. When using sensors fabricated from T, P and H as well as their 1:1 mixtures (100 mM), the presence of AP in the EtOH solution was difficult to detect in the specific RGB intensity plots (FIGS. 7a-d) but it could be identified in the PC analysis. Similar to solutions supplemented with EA, only the presence of AP, was clear but the different amounts could not be distinguished. The different concentrations of AP became better separable when sensors consisting of T, T/P and P with lower AP concentrations were used (20, 50, 75 mM). While the variations in the sensor responses were barely visible in the scanned images, the PC analysis revealed distinct cluster means for 5% EtOH solutions and 5% EtOH solutions supplemented with 2 and 19 ppm AP and 186 ppm AP (FIG. 7e). 2 and 10 ppm AP gave a similar sensor response.

### Example 8—Mapping Beers

Example 8 assesses the feasibility of the paper-based PDA sensors to distinguish four different commercial beer types.

Three beers with increasing amounts of EtOH were chosen: Carlsberg Nordic: (CB N, 0.5% EtOH), Carlsberg Classic (CB C, 4.6% EtOH) and Wiibroe Årgangsøl (WB, 10.6% EtOH). Further, Tuborg Classic (TB C, 4.6% EtOH) was added to the list in order to ensure that the detected colour differences were not solely due to the different EtOH contents (i.e. since it has the same alcohol content as Carlsberg Classic).

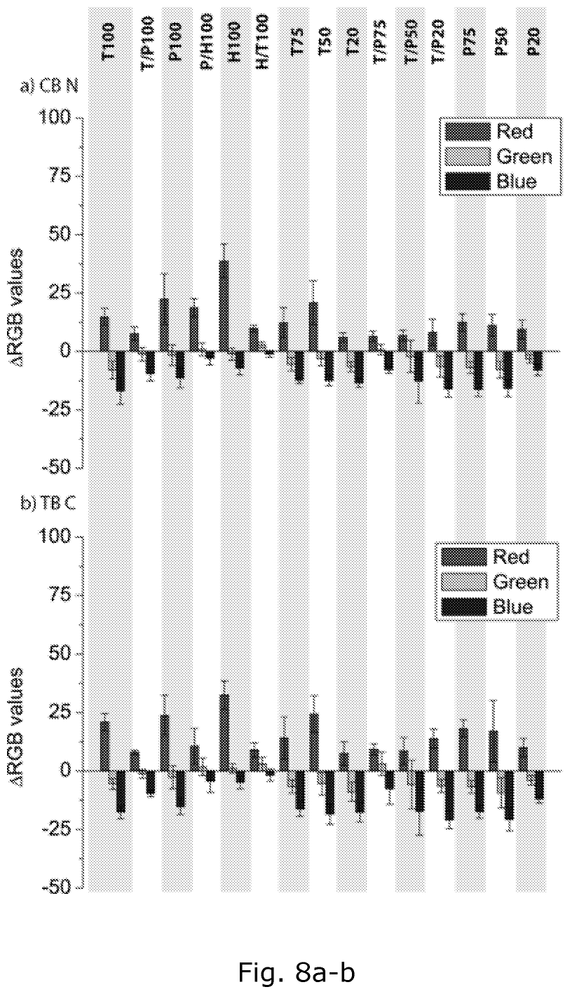

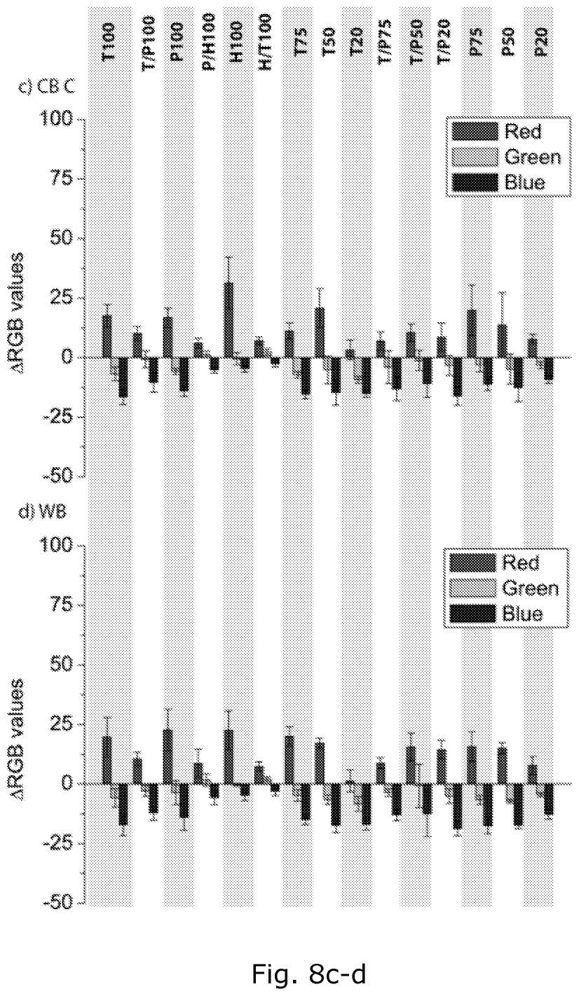

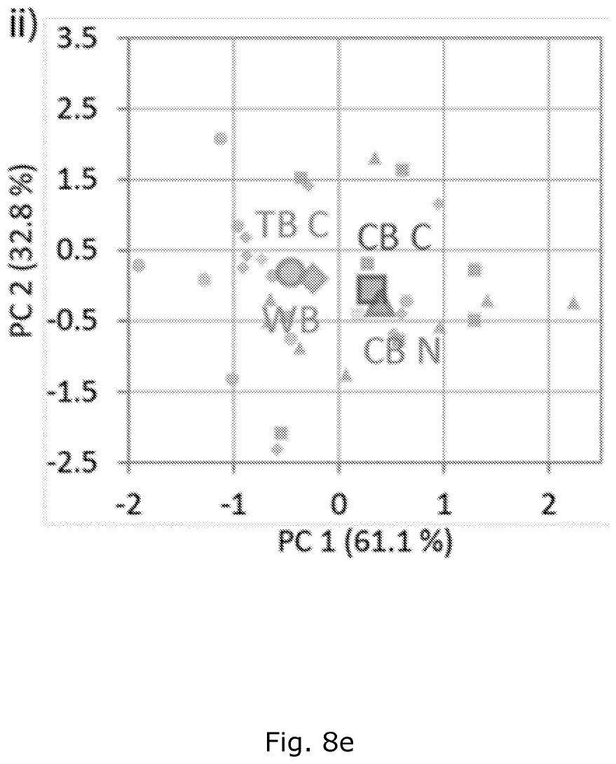

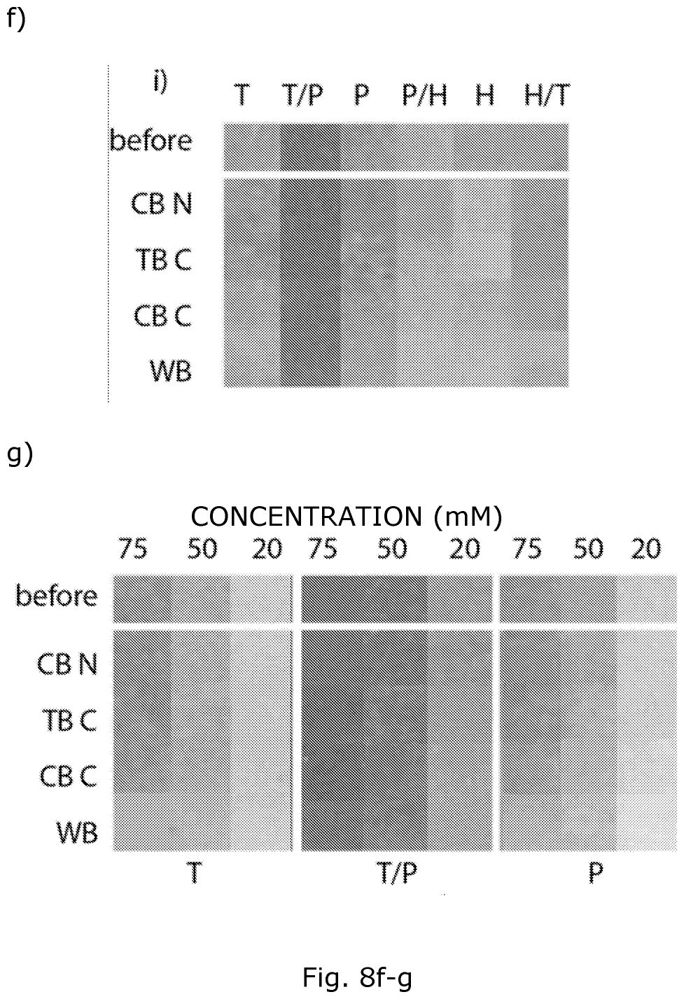

First, sensors fabricated from T, P and H as well as their 1:1 mixtures (100 mM) responded in similar way when exposed to the four beers as seen in the specific RGB intensity plots (FIGS. 8a-d)). The different beer types became separable when sensors consisting of T, T/P and P with lower DA concentrations (20, 50, 75 mM) were used (FIGS. 8f-g). Although the visual changes in the sensors were minor, the PC analysis revealed beer dependent different cluster means (FIG. 8e).

Importantly, the variations were not exclusively caused by the different EtOH contents, since CB C and TB C had both the same EtOH content by distinguishable clusters means. On a side note, the sensors were not able to separate CB C and CB N, pointing towards the successful preservation of the composition in the non-alcoholic version.

### Example 9—Formation of Pda Vesicles in Solution

PDA vesicles in solution may be produced in accordance with the procedure in FIG. 9. Arrays may be obtained using e.g. well plates, with a sensor solution in each well.

Generally DA monomers are dissolved in a suitable amount of solvent, such as chloroform. The solvent is thereafter evaporated in a flask to produce a thin film on the inside of the flask. The thin film is hydrated and sonicated to produce a film dispersion which is subjected to extrusion and co-assembly. The unpolymerised vesicles formed are treated with UV irradiation to form polymerised blue vesicles.

Solutions were produced using mixtures of TCDA (T) and TCDA-PEG monomers as provided in Table 2 entry 14-16. PEGs used were e.g. PEG550, PEG4 and mPEG. The TDCA to TDCA-PEG ratio was 4:6.

### Example 10—Solution Measurements on Selected Analytes with Specific Pda Vesicles

### Esters, Alcohols and 4-VG

Solution based sensors made from the following DA monomer mixtures were tested with a number of analytes including alcohols, esters and 4-VG:

| | |
|---|---|
| 1) TDCA (T), | (Table 2, No. 3) |
| 2) TDCA (T) + TDCA-SH | (Table 2, No. 3 + 18) |
| 3) TDCA (T) + TDCA-PEG4 | (Table 2, No. 3 + 14) |

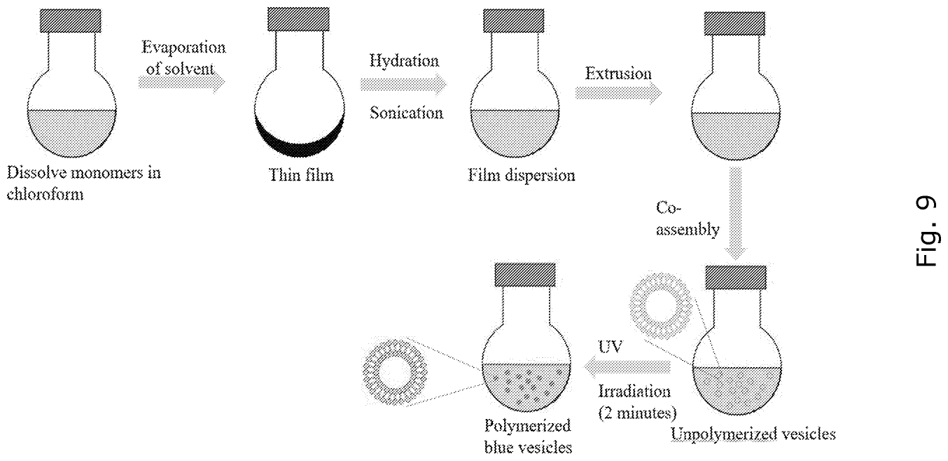

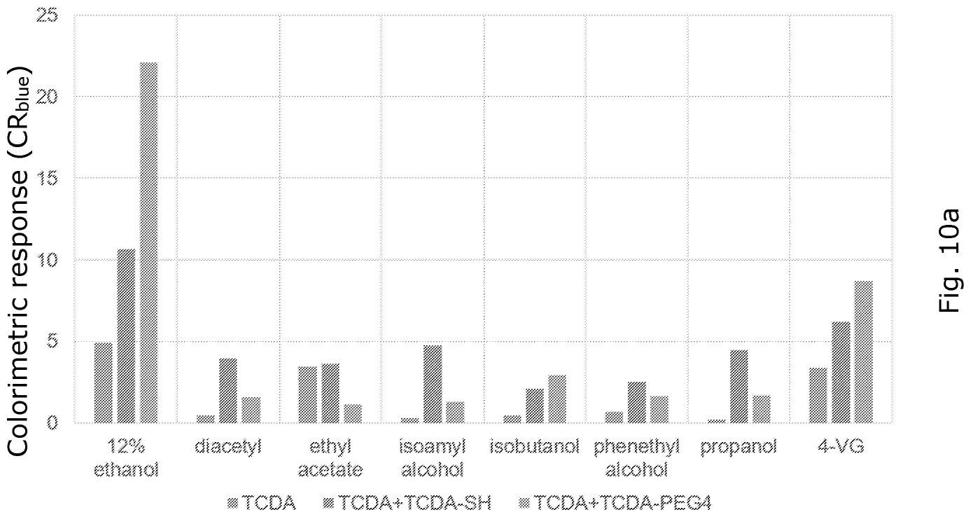

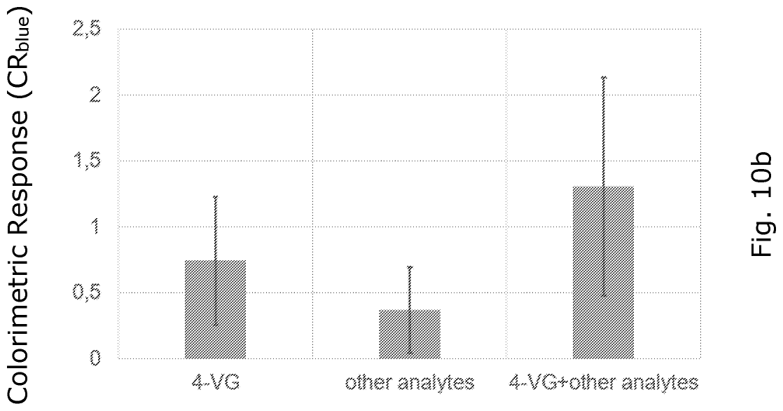

The results in the form of the colorimetric response (CR_{blue}) determined as described under “detection” above are shown in FIG. 10a for ethanol, DAc, EA, isoamyl alcohol, isobutanol, phenethyl alcohol, propanol, and 4-VG. The suitability of the solution arrays is demonstrated by the responses being present and different for each sensor and each analyte. As 4-VG is an analyte of particular interest it was tested whether it could be detected when mimicking the beer environment, i.e. in the presence of water, ethanol and further analytes, the further analytes being those represented in FIG. 10a. As seen in FIG. 10b, 4-VG was readily detectable both alone and in the presence of other analytes. Sensor 3) above was used in this experiment. Further PDA sensors were tested, which were polymerised from the monomers of table 2 or mixtures thereof, and it showed the suitability of these sensors in providing responses to the different analytes, also in the presence of other analytes.

### Example 11—Comparative Study Using Reference and Test Assay

Three essentially identical arrays are fabricated from T, P and H the sensors of table 2 above as well as mixtures thereof (75 mM). They are produced on paper support. The arrays are produced using the same method and the same PDA in each position on the array. The same DA solution is used for each sensor on all arrays.

For the particular arrays used in the experiments related to FIGS. 11a-b the following PDA sensors were used:

TABLE 3
PDA sensors used in Example 11, Figures 11a-b

| | PDA | | |
|---|---|---|---|
| Sensor no | composition | | Total PDA |
| (Figure | (table 2 | Molar | concentration |
| 11a-b) | numbering) | ratio | (mM) |
| Sensor 1 | 2 | — | 75 |
| Sensor 4 | 2 + 3 | 1:1 | 75 |
| Sensor 7 | 3 | — | 75 |
| Sensor 13 | 4 | — | 75 |
| Sensor 34 | 2 + 4 | 1:1 | 75 |
| Sensor 25 | 5 | — | 75 |
| Sensor 28 | 4 + 5 | 1:1 | 75 |
| Sensor 22 | 2 + 4 | 4:1 | 75 |
| Sensor 38 | 1 + 2 | 1:1 | 75 |
| Sensor 39 | 2 + 9 | 1:1 | 75 |

The first array (the reference array) is subjected to e.g. a commercially available beer, a production batch, or a beer precursor to produce a colorimetric response, which could be measured by first reading out the RGB values of a sensor as produced and after exposure to the analyte solution. In addition to using the RGB changes for principal component analysis, these RGB values can be used to determine the Hue change (ΔHue) or the red chromaticity shift (RCS). These values can then be categorized (e.g., non-change, weak change, strong change) and graphically represented as e.g., pie charts, representing simple fingerprints of reference and test samples (see FIG. 11a-b, where 4 commercially available beers have been tested against arrays of 10 sensors (table 3), and where ΔHue and RCS values are presented as colours, i.e. darker colours representing higher values).

The second essentially identical array (the 1st test array) is subjected to the same commercial beer, production batch, or beer precursor following the same procedure as for the reference array. The results show that the reference array and 1^{st} test array produce a highly similar colorimetric response.

The third essentially identical array (the 2^{nd} test array) is subjected to a different commercially available beer, production batch, or beer precursor of the same type and alcohol percentage as the initial commercially available beer. The 2^{nd} test array produce a response which is markedly different from the reference array, as seen in FIGS. 11a-b.

These results show the ability of the sensor array system to identify similar beers and different beers very clearly. This can further include comparison of e.g. new batches with previous successful batches.

### Example 12—Synthetic Procedures Towards Da Monomers

Depending on the structures of the DA monomers, different synthetic protocols were employed.

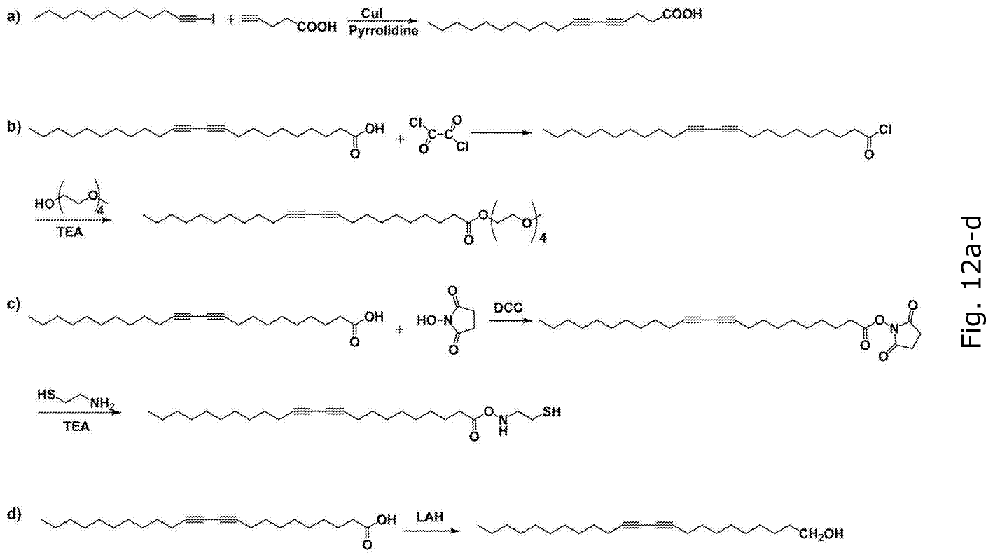

For monomers 4, 5, 6 with similar structures to H, T and P, a classical Cadiot-Chodkiewicz coupling reaction, starting with an acetylene and a halo-acetylene, catalysed by Cu(I) in the presence of an amine as the base was utilized, as shown in FIG. 12a (taking monomer 4 as an example).

For monomers 7, 14, 15, 16, 17 with ester groups, an esterification reaction between acyl chloride and alcohol was employed. Acyl chloride was obtained by treating DA monomer T with oxalyl chloride (FIG. 12b, taking monomer 14 as an example).

For monomers 9, 10, 11, 12, 13, 18, 19, 20, 21 with amide groups, the DA precursors T or P were first treated with N-Hydroxysuccinimide (NHS) and N,N′-Dicyclohexylcarbodiimide (DCC) to obtain NHS ester, which subsequently reacted with the corresponding amine-containing precursor in the presence of triethylamine (TEA) to obtain the final DA monomers, as illustrated in FIG. 12c, taking monomer 18 as an example.

Monomer 8 was synthesised by the reduction of DA monomer T promoted by lithium aluminium hydride (LAH), as shown in FIG. 12d.

The structure of the monomers could be confirmed using H-NMR, C-NMR and mass spectroscopy.

The synthesised DA monomers have been subject to preliminary testing showing good stability, polymerizability and colorimetric responses to various analytes. Ionic DA monomers (e.g. imidazolium salts) are advantageous in solution phase vesicle formation.

### CONCLUSIONS

Herein, we report the assembly of PDA sensors on a paper substrate and vesicles in solution to distinguish different types of beers. The sensor composition and the DA concentration used during the fabrication were factors in tailoring the efficiency of the sensor to detect different EtOH and other analyte concentrations, analytes in an EtOH/water solution and differentiate different beers. In general, lowering the concentration of T and P during the sensor assembly contributed to the selectivity. These sensors allowed for the distinction of EtOH solutions and the identification of down to 10 ppm of EA, DAc ad AP in a 5% EtOH solution.

### REFERENCES

EP 2947455 A1
US2016/0061741 A1
EP 1161688 B1
Eaidkong T. et al., J. Mater. Chem., 2012, 22, 5970
M. Roman and M. Baranska, spectrochim. Acta Mol. Biomol. Spectrosc., 2015, 127, 652.

1-20. (canceled)

21. A method for characterizing an aqueous solution for at least one analyte, comprising the steps of:
a) providing a sensor array comprising at least two different poly-diacetylenes, wherein said poly-diacetylenes are spatially separated and individually addressable,
b) contacting said sensor array with a sample of said aqueous solution,
c) measuring the colorimetric response of said poly-diacetylenes to the aqueous solution,
wherein said poly-diacetylenes are polymerized from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, wherein
said poly-diacetylenes are capable of a colorimetric response upon contact with said analyte, and wherein
the at least one analyte is selected from the group consisting of an organic molecule with a molecular weight below 2000 g/mol, salts thereof and an inorganic salt
wherein the aqueous solution is a beverage or precursor thereof.

22. The method according to claim 21, wherein said one or more diacetylene monomer(s) is selected from the group of diacetylenes according to formula (I) or (II) 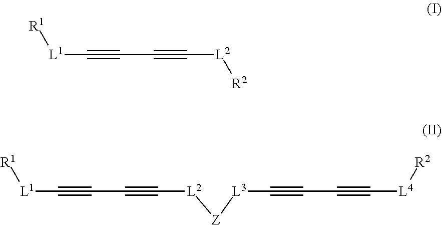
or mixtures thereof, wherein
L¹, L², L³ and L⁴ are the same or different and individually selected from the group consisting of an optionally substituted C₁-C₃₀ alkylene, an optionally substituted C₂-C₃₀ alkenylene, and an optionally substituted C₂-C₃₀ alkynylene,
R¹ and R² are the same or different and individually selected from the group consisting of —CH₃, OR³, SR³, —COOR³, —CONR⁴R⁵, wherein
R³, R⁴, and R⁵ are individually selected from the group consisting of hydrogen, C₁-C₈ alkyl optionally substituted with a thiol, vinyl, or optionally substituted imidazolium, and a polyethylene glycol alkyl ether optionally substituted with a thiol, vinyl, or optionally substituted imidazolium, or are selected so that NR⁴R⁵ constitutes an amino acid,
Z is selected from the group consisting of optionally substituted alkylene, aryl, —CONH—(CH₂)x-HNCO— where X is an integer between 1 and 20, and heteroaryl.

23. The method according to claim 22, wherein L¹, L², L³ and L⁴ are the same or different and individually selected from a —(CH₂)ₙ— group wherein n is 1-30.

24. The method according to claim 22, wherein R¹ and R² are the same or different and individually selected from the group consisting of —CH₃, and —COOR³.

25. The method according to claim 22, wherein R³, R⁴, and R⁵ are individually selected from the group consisting of hydrogen, and C₁-C₃ alkyl.

26. The method according to claim 22, wherein
L¹, L², L³ and L⁴ are the same or different and individually selected from a —(CH₂)ₙ— group wherein n is 1-20,
R¹ and R² are the same or different and individually selected from the group consisting of —CH₃, —COOR³, —CONR⁴R⁵, wherein R³, R⁴, and R⁵ are individually selected from the group consisting of hydrogen, and C₁-C₈ alkyl optionally substituted with a thiol, vinyl, or optionally substituted imidazolium, and a polyethylene glycol alkyl ether optionally substituted with a thiol, vinyl, or optionally substituted imidazolium, or are selected so that NR⁴R⁵ constitutes an amino acid, and
Z is selected from the group consisting of optionally substituted alkylene, aryl, —CONH—(CH₂)x-HNCO— where X is an integer between 1 and 20, and heteroaryl.

27. The method according to claim 21, wherein the one or more diacetylene monomer(s) is selected from the group of diacetylenes according to formula (I) 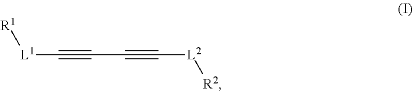
wherein
L¹ is a —(CH₂)ₙ— group wherein n is an integer in the range of 1 to 20; and
L² is a —(CH₂)ₙ— group wherein n is an integer in the range of 1 to 20; and
R¹ and R² are the same or different and individually selected from the group consisting of —CH³, —COOR³, —CONR⁴R⁵, wherein
R³, R⁴, and R⁵ are individually selected from the group consisting of hydrogen, and C₁-C₈ alkyl optionally substituted with a thiol, vinyl, an amino acid, or optionally substituted imidazolium, and a polyethylene glycol alkyl ether optionally substituted with a thiol, vinyl, an amino acid, or optionally substituted imidazolium, or are selected so that NR⁴R⁵ constitutes an amino acid.

28. The method according to claim 27, wherein the one or more diacetylene monomer(s) is selected from the group of diacetylenes according to formula (I), wherein
L¹ and L² are a —(CH₂)ₙ— group wherein n is an integer in the range of 1 to 12,
R¹ and R² are the same or different and individually selected from the group consisting of —CH³, —COOR³, —CONR⁴R⁵, wherein
R³, R⁴, and R⁵ are individually selected from the group consisting of hydrogen, and C₁-C₈ alkyl optionally substituted with a thiol, vinyl, an amino acid, or optionally substituted imidazolium, and a polyethylene glycol alkyl ether optionally substituted with a thiol, vinyl, an amino acid, or optionally substituted imidazolium, or are selected so that NR⁴R⁵ constitutes an amino acid.

29. The method according to claim 21, wherein at least one of the poly-diacetylenes is a polymer polymerized from a mixture comprising at least two different diacetylene monomers.

30. The method according to claim 21, wherein the concentration of diacetylene monomer or mixture thereof during polymerisation is in the range of 1-1000 mM.

31. The method according to claim 21, wherein the at least one analyte is a flavour constituent of a beverage.

32. The method according to claim 31, wherein the beverage is beer.

33. The method according to claim 31, wherein the flavour constituent present of the beverage is selected from the group consisting of ethanol, carbonic acid, hop bitter substances, hop oil constituents, maltol, monosaccharides, disaccharides, banana esters, apple esters, 3-methylbutanol, dimethyl sulfide, C₆-C₁₂ fatty acids, acetic acid, propanoic acid, ethyl acetate, 2,3-butanedione, citric acid, maleic acid, polyphenols, trisaccharides, amino acids, diacetyl, acetylpropionyl, acetaldehyde, isobutylacetate, propanol, isobutanol, isoamylacetate, isoamylalcohol, ethyl caproate, ethyl caprylate, 2-phenylethylaceteate, caprylic acid, caproic acid, capric acid, linalool, limonene, pentanedione, λ-decalactone, 2-phenylethanol, trans-2-noneal, 4-vinylguaiacol (4-VG), hydrogen sulfide, 3-methyl-2-butene-1-thiol, and sodium chloride.

34. The method according to claim 33, wherein the at least one flavour constituent is selected from the group consisting ethanol, ethyl acetate, diacetyl, 4-vinylguaiacol, an apple ester wherein the apple ester comprises ethyl hexanoate, isoamylacetate, and acetylpropionyl.

35. The method according to claim 21, wherein the beverage is selected from the group consisting of beer, cider, white wine, rosé wine, red wine, dairy products, soft-drinks, alcopops and precursors thereof.

36. The method according to claim 21, wherein the at least two different poly(diacetylene) polymers are positioned in a vesicle or micelle.

37. The method according to claim 21, wherein the at least two different poly(diacetylene) polymers are positioned on a solid support.

38. The method according to claim 21, wherein there are at least 3 different spatially separated poly(diacetylene) polymers.

39. The method according to claim 21, wherein said method is capable of differentiating distinct beers or beer precursors.

40. A method for characterizing a beer or a beer precursor for multiple analytes, comprising the steps of:
a) providing a sensor array comprising at least two different poly-diacetylenes, wherein said poly-diacetylenes are spatially separated and individually addressable,
b) contacting said sensor array with a sample of a beer or a beer precursor,
c) measuring the colorimetric response of said poly-diacetylenes to the beer or beer precursor, and
wherein said poly-diacetylenes are polymers polymerised from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, and
wherein said sensor array for each analyte comprises at least one poly-diacetylene capable of a colorimetric response to contact to said analyte, and
wherein the analytes are flavour constituents of beer.

41. A method for comparing a test aqueous solution with a reference aqueous solution comprising at least one analyte, comprising the steps of:
a) providing at least two identical sensor arrays comprising at least two different poly-diacetylenes, wherein said poly-diacetylenes are spatially separated and individually addressable,
b) contacting a first sensor array with a sample of the test aqueous solution and a second sensor array with a reference aqueous solution,
c) comparing the colorimetric response of said poly-diacetylenes of the first sensor array to the colorimetric response of said poly-diacetylenes of the second sensor array,
wherein a similar colorimetric response of the first sensor array and the second sensor array indicates that the test aqueous solution is similar to the reference aqueous solution; and
wherein said poly-diacetylenes are polymers polymerised from a composition comprising one or more diacetylene monomer(s), said diacetylene monomer(s) comprising one or more substituent(s) selected from the group consisting of an optionally substituted C₁-C₃₀ alkyl, an optionally substituted C₂-C₃₀ alkenyl, and an optionally substituted C₂-C₃₀ alkynyl, wherein
said poly-diacetylenes are capable of a colorimetric response upon contact with said analyte, and
wherein the at least one analyte is selected from the group consisting of an organic molecule with a molecular weight below 2000 g/mol, salts thereof and an inorganic salt, and
wherein the aqueous solution is a beverage or precursor thereof.
